(19) United States
(12) Patent Application Publication
(10) Pub. No.: US 2022/0105113 A1
(43) Pub. Date: Apr. 7, 2022

(54) CYLODIMER OF DEHYDROSALICORTIN AND DERIVATIVES THEREOF ISOLATED FROM PLANT OF THE GENUS SALIX FOR USE IN CANCER THERAPY

(71) Applicants: ROTHAMSTED RESEARCH LTD, Harpenden Hertfordshire (GB); UNIVERSITY OF KENT, Canterbury, Kent (GB)

(72) Inventors: Michael Henry BEALE, Harpenden, Hertfordshire (GB); Jane Louise WARD, Hertfordshire (GB); Yanqi WU, Harpenden, Hertfordshire (GB); Martin MICHAELIS, Canterbury Kent (GB)

(73) Assignees: ROTHAMSTED RESEARCH LTD, Harpenden, Hertfordshire (GB); UNIVERSITY OF KENT, Canterbury Kent (GB)

(21) Appl. No.: 17/426,424

(22) PCT Filed: Jan. 29, 2020
(86) PCT No.: PCT/GB2020/050203

§ 371 Date: Jul. 28, 2021

(30) Foreign Application Priority Data
Jan. 30, 2019 (GB) 1901272.3
Oct. 10, 2019 (GB) 1914640.6

(51) Int. Cl. A61K 31/704

(52) U.S. Cl. CPC A61K 31/704

(57) ABSTRACT

Described herein are compounds comprising a dimer of dehydrosalicortin or a derivative, homologue, stereoisomer, prodrug or pharmaceutical salt thereof. In particular embodiments, the dimer is a result of a Diels-Alder reaction. Also described are compositions comprising the compounds and their use in treating disease.

The present invention relates to novel compounds and their use in therapy, in particular for the treatment of cancer.

Cancer is a disease which effects millions of people around the world each year. Whilst many effective therapies exist for treating cancer, there are still a huge number of cancers for which there is either no treatment or for which current treatments remain largely ineffective. This, combined with the large number of different types of cancer now known, means that there is a great need for new therapies.

It is, therefore, an object of the present invention to seek to alleviate the above identified problem.

### SUMMARY OF THE INVENTION

According to one aspect of the present invention, there is provided a compound comprising a dimer of dehydrosalicortin or a derivative, homologue, stereoisomer, prodrug or pharmaceutical salt thereof.

Preferably, the dimer is a result of a Diels-Alder reaction.

According to another aspect of the present invention, there is provided a compound of Formula I, or a derivative, homologue, stereoisomer, prodrug or pharmaceutical salt thereof.

Within this specification, reference to a compound of Formula I means a compound having the following structure:—

R¹-L-R² Formula I
wherein L is a linking member, R¹ and R² are each independently selected from Formula III.

Within this specification, reference to Formula III means:—

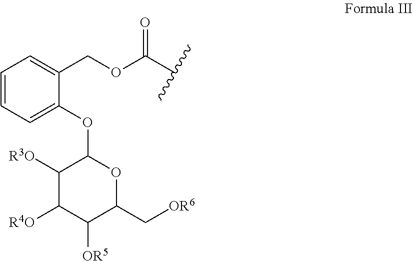

wherein R³, R⁴, R⁵ and R⁶ are each independently selected from (i) H, (ii) acetyl, (iii) benzoyl, (iv) ortho- or para-coumaroyl, (v) cinnamoyl.

Preferably, R³, R⁴, R⁵ and R⁶ are each independently selected from (i) H, and (ii) acetyl.

Preferably, R³, R⁴, R⁵ and R⁶ are each H.

For the sake of convenience, Table 1 provides structures for some of the groups referred to herein.

TABLE 1

| Term | Structure |
|---|---|
| acetyl | 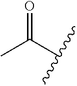 [SMILES: *C(C)=O] |
| benzoyl | 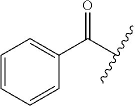 [SMILES: *C(=O)c1ccccc1] |
| para-coumaroyl | 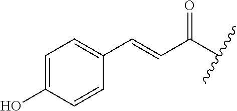 [SMILES: *C(=O)/C=C/c1ccc(O)cc1] |
| ortho-coumaroyl | 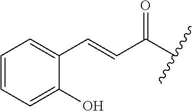 [SMILES: *C(=O)/C=C/c1ccccc1O] |
| cinnamoyl | 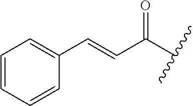 [SMILES: *C(=O)/C=C/c1ccccc1] |

Preferably, R¹ is Formula IIIA:—

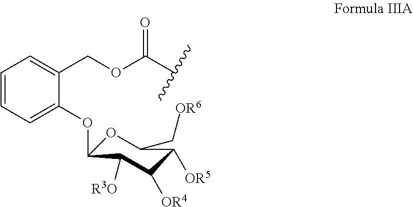

wherein R³, R⁴, R⁵ and R⁶ are each independently selected from (i) H, (ii) acetyl, (iii) benzoyl, (iv) ortho- or para-coumaroyl, (v) cinnamoyl.

Preferably, R³, R⁴, R⁵ and R⁶ are each independently selected from (i) H, and (ii) acetyl.

Preferably, R³, R⁴, R⁵ and R⁶ are each H.

Preferably, R¹ is Formula IIIB—

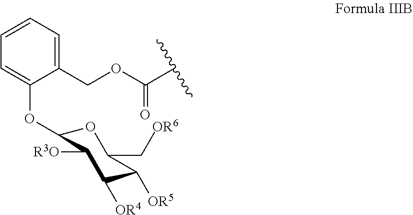

wherein R³, R⁴, R⁵ and R⁶ are each independently selected from (i) H, (ii) acetyl, (iii) benzoyl, (iv) ortho- or para-coumaroyl, (v) cinnamoyl. Preferably, R³, R⁴, R⁵ and R⁶ are each independently selected from (i) H, and (ii) acetyl. Preferably, R³, R⁴, R⁵ and R⁶ are each H.

Preferably, R² is Formula IIIC:—

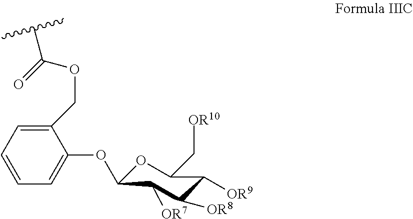

wherein R⁷, R⁸, R⁹ and R¹⁰ are each independently selected from (i) H, (ii) acetyl, (iii) benzoyl, (iv) ortho- or para-coumaroyl, (v) cinnamoyl.

Preferably, R⁷, R⁸, R⁹ and R¹⁰ are each independently selected from (i) H, and (ii) acetyl.

Preferably, R⁷, R⁸, R⁹ and R¹⁰ are each H.

Preferably, R² is Formula IIID:—

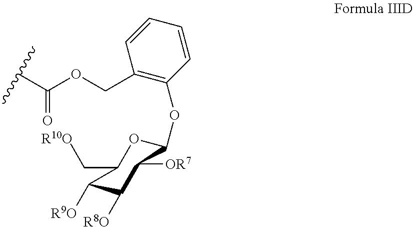

wherein R⁷, R⁸, R⁹ and R¹⁰ are each independently selected from (i) H, (ii) acetyl, (iii) benzoyl, (iv) ortho- or para-coumaroyl, (v) cinnamoyl.

Preferably, R⁷, R⁸, R⁹ and R¹⁰ are each independently selected from (i) H, and (ii) acetyl.

Preferably, R⁷, R⁸, R⁹ and R¹⁰ are each H.

Preferably, L comprises a ring structure.

Preferably, L is the result of a Diels-Alder reaction.

Preferably, L comprises a Diels-Alder reaction produced core element.

Preferably, L comprises a tricyclododecadiene derivative, preferably a substituted derivative, preferably wherein the cycloalkene is substituted by at least one group selected from OH, carbonyl.

Preferably, L is selected from Formula IIA or Formula IIB.

Within this specification, reference to Formula IIA means:—

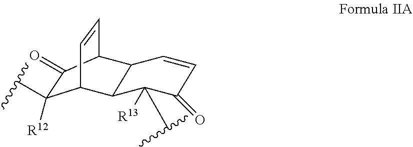

wherein R¹² and R¹³ are each independently selected from H and OH.

Preferably, R¹² and R¹³ are each OH.

Within this specification, reference to Formula IIB means:—

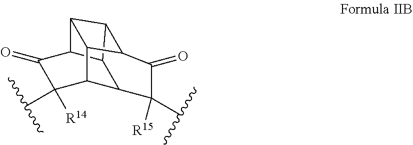

wherein R¹⁴ and R¹⁵ are each independently selected from H and OH.

Preferably, R¹⁴ and R¹⁵ are each OH.

According to another aspect of the present invention, there is provided a compound of Formula IIC, or a derivative, homologue, stereoisomer, prodrug or pharmaceutical salt thereof.

Within this specification, reference to a compound of Formula IIC means a compound having the following structure:—

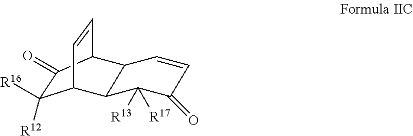

wherein R¹⁶ and R¹⁷ are each independently selected from H, OH and Formula III, with the proviso that at least one of R¹⁶ and R¹⁷ is selected from Formula III, and
wherein R¹² and R¹³ are each independently selected from H and OH.

Preferably, R¹⁶ and R¹⁷ are selected from Formula III.

Preferably, either R¹⁶ or R¹⁷ is selected from Formula III.

Preferably, R¹⁶ is H and R¹⁷ is selected from Formula III.

Preferably, R¹² and R¹³ are each OH.

According to another aspect of the present invention, there is provided a compound of Formula IID, or a derivative, homologue, stereoisomer, prodrug or pharmaceutical salt thereof.

Within this specification, reference to a compound of Formula IID means a compound having the following structure:—

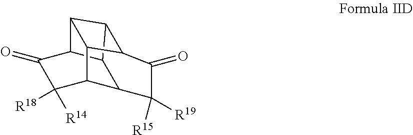

wherein R¹⁸ and R¹⁹ are each independently selected from H, OH and Formula III, with the proviso that at least one of R¹⁸ and R¹⁹ is selected from Formula III, and
wherein R¹⁴ and R¹⁵ are each independently selected from H and OH.

Preferably, R¹⁸ and R¹⁹ are selected from Formula III.

Preferably, either R¹⁸ or R¹⁹ is selected from Formula III.

Preferably, R¹⁴ and R¹⁵ are each OH.

Preferably, the invention relates to a compound of Formula VII or a derivative, homologue, stereoisomer, prodrug or pharmaceutical salt thereof.

Within this specification, reference to Formula VII means:—

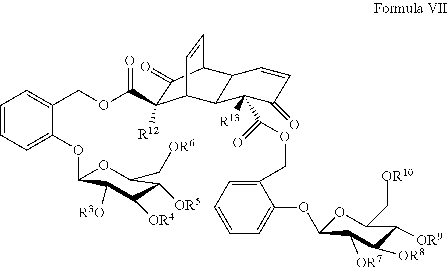

wherein R³, R⁴, R⁵, R⁶, R⁷, R⁸, R⁹ and R¹⁰ are each independently selected from (i) H, (ii) acetyl, (iii) benzoyl, (iv) ortho or para-coumaroyl, (v) cinnamoyl, and
wherein R¹² and R¹³ are each independently selected from H and OH.

Preferably, R³, R⁴, R⁵, R⁶, R⁷, R⁸, R⁹ and R¹⁰ are each independently selected from (i) H, and (ii) acetyl.

Preferably, R³, R⁴, R⁵, R⁶, R⁷, R⁸, R⁹ and R¹⁰ are each H.

Preferably, R¹² and R¹³ are each OH.

Preferably, the invention relates to a compound of Formula VIII or a derivative, homologue, stereoisomer, prodrug or pharmaceutical salt thereof.

Within this specification, reference to Formula VIII means:—

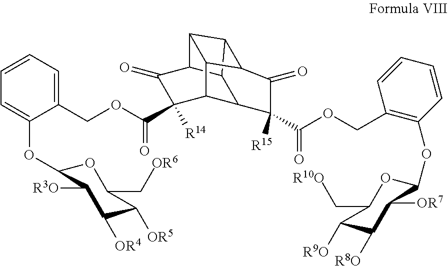

wherein R³, R⁴, R⁵, R⁶, R⁷, R⁸, R⁹ and R¹⁰ are each independently selected from (i) H, (ii) acetyl, (iii) benzoyl, (iv) ortho- or para-coumaroyl, (v) cinnamoyl, and
wherein R¹⁴ and R¹⁵ are each independently selected from H and OH.

Preferably, R³, R⁴, R⁵, R⁶, R⁷, R⁸, R⁹ and R¹⁰ are each independently selected from (i) H, and (ii) acetyl.

Preferably, R³, R⁴, R⁵, R⁶, R⁷, R⁸, R⁹ and R¹⁰ are each H.

Preferably, R¹⁴ and R¹⁵ are each OH.

Preferably, the invention relates to a compound of Formula IX, or a derivative, homologue, stereoisomer, prodrug or pharmaceutical salt thereof.

Within this specification, reference to Formula IX means:—

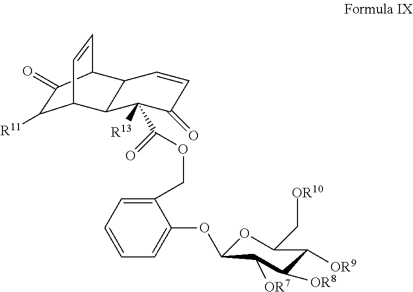

wherein R⁷, R⁸, R⁹ and R¹⁰ are each independently selected from (i) H, (ii) acetyl, (iii) benzoyl, (iv) ortho- or para-coumaroyl, (v) cinnamoyl,
wherein R¹¹ is selected from (i) H, (ii) OH, and (iii) Formula III.
wherein R¹³ is selected from H and OH.

Preferably, R⁷, R⁸, R⁹ and R¹⁰ are each independently selected from (i) H, and (ii) acetyl.

Preferably, R⁷, R⁸, R⁹ and R¹⁰ are each H. Preferably, R¹³ is H.

Preferably R¹¹ is H or OH. Preferably R¹¹ is OH.

Preferably, the invention relates to a compound of Formula X or a derivative, homologue, stereoisomer, prodrug or pharmaceutical salt thereof.

Within this specification, reference to Formula X means:—

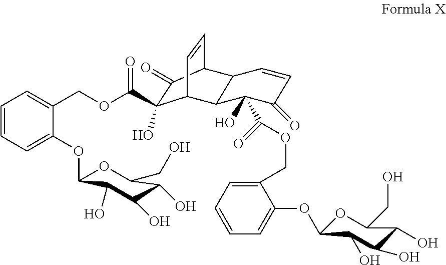

Preferably, the invention relates to a compound of Formula XI, or a derivative, homologue, stereoisomer, prodrug or pharmaceutical salt thereof.

Within this specification, reference to Formula XI means:—

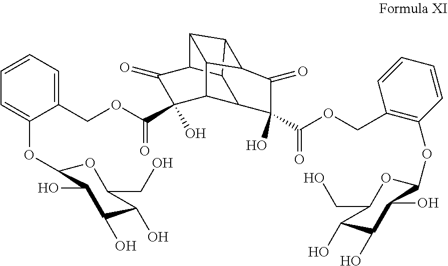

Preferably, the invention relates to a compound of Formula XII, or a derivative, homologue, stereoisomer, prodrug or pharmaceutical salt thereof.

Within this specification, reference to Formula XII means:—

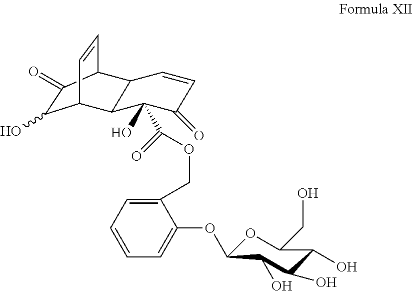

Preferably, the invention relates to a compound of Formula XIII or a derivative, homologue, stereoisomer, prodrug or pharmaceutical salt thereof.

Within this specification, reference to Formula XIII means:—

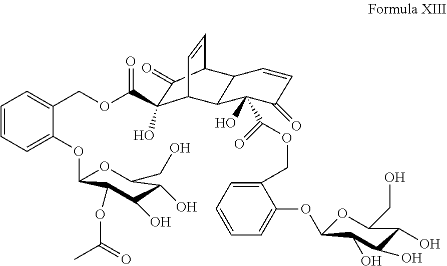

Preferably, the invention relates to a compound of Formula XIV or a derivative, homologue, stereoisomer, prodrug or pharmaceutical salt thereof.

Within this specification, reference to Formula XIV means:—

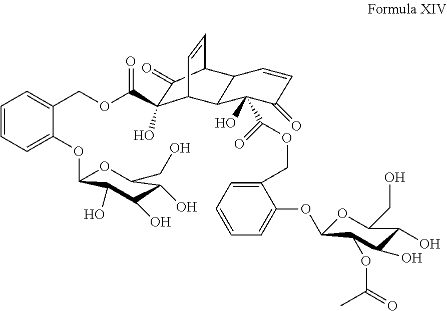

Preferably, the invention relates to a compound of Formula XV or a derivative, homologue, stereoisomer, prodrug or pharmaceutical salt thereof.

Within this specification, reference to Formula XV means:—

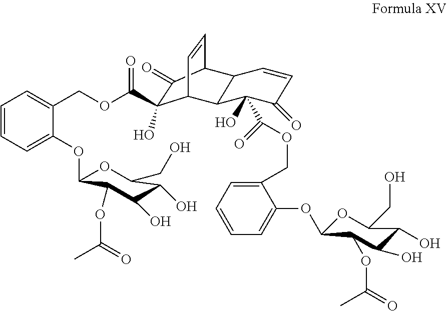

Preferably, the invention relates to a compound of Formula XVI or a derivative, homologue, stereoisomer, prodrug or pharmaceutical salt thereof.

Within this specification, reference to Formula XVI means:—

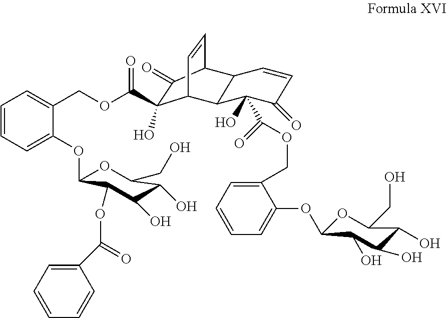

Preferably, the invention relates to a compound of Formula XVII or a derivative, homologue, stereoisomer, prodrug or pharmaceutical salt thereof.

Within this specification, reference to Formula XVII means:—

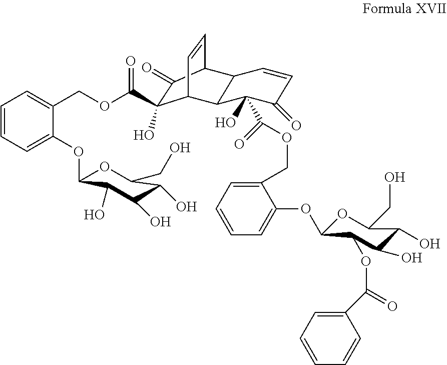

Preferably, the invention relates to a compound of Formula XVIII or a derivative, homologue, stereoisomer, prodrug or pharmaceutical salt thereof.

Within this specification, reference to Formula XVIII means:—

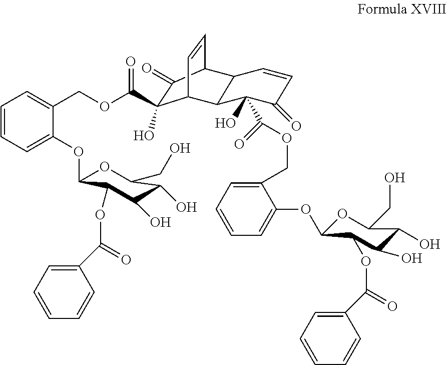

According to another aspect of the present invention, there is provided a compound of Formula XIX or a derivative, homologue, stereoisomer, prodrug or pharmaceutical salt thereof.

Within this specification, reference to Formula XIX means:—

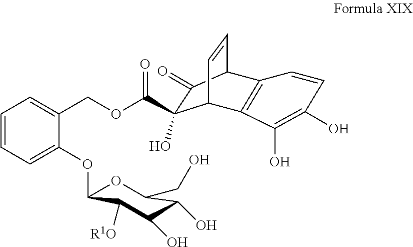

wherein R¹ is selected from (i) H, (ii) acetyl, (iii) benzoyl, (iv) ortho- or para-coumaroyl, (v) cinnamoyl.

Preferably, the invention relates to a compound of Formula XX or a derivative, homologue, stereoisomer, prodrug or pharmaceutical salt thereof.

Within this specification, reference to Formula XX means:—

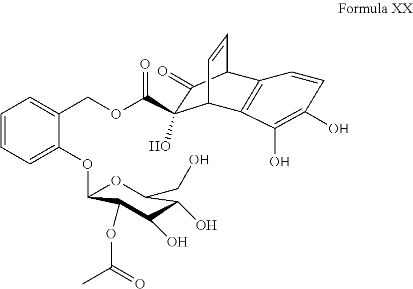

Preferably, the derivative, homologue, stereoisomer, prodrug or pharmaceutical salt thereof is a therapeutically effective derivative, homologue, stereoisomer, prodrug or pharmaceutical salt thereof.

According to another aspect of the present invention, there is provided a composition comprising a compound as described herein, or a derivative, homologue, stereoisomer, prodrug or pharmaceutical salt thereof.

Preferably, the composition comprises a compound of Formula VII, VIII or IX, most preferably a compound of Formula VII, or a derivative, homologue, stereoisomer, prodrug or pharmaceutical salt thereof.

Preferably, the composition comprises a compound of Formula X, XI or XII, most preferably a compound of Formula X, or a derivative, homologue, stereoisomer, prodrug or pharmaceutical salt thereof.

Preferably, the composition is a pharmaceutical composition.

Preferably, the composition is a therapeutic composition.

Preferably, the composition comprises one or more pharmaceutically acceptable carriers, diluents or excipients.

According to another aspect of the present invention, there is provided a composition as described herein for use in therapy.

According to a further aspect of the present invention, there is provided use of a composition as described herein for treating a disease.

According to another aspect of the present invention, there is provided use of a composition as described herein in the manufacture of a medicament for treating a disease.

According to a further aspect of the present invention, there is provided a method of treating a disease, wherein the method comprises administering to a patient suffering from a disease a therapeutically effective amount of a composition as described herein.

Preferably, therapy comprises treating a disease.

Preferably, treating a disease comprises administering a therapeutically effective amount of a composition of the present invention to a patient suffering from a disease.

Preferably, the disease is cancer.

Preferably, the cancer is primary or secondary (metastatic) cancer.

Preferably, the cancer is a drug-resistant cancer. In this respect, it will be appreciated that reference to “drug resistant cancer” means a cancer which has previously shown resistance to treatment with another therapeutic composition, for example a cancer which has been unsuccessfully treated with another therapeutic composition.

Preferably, the cancer is resistant to an anti-microtubule agent, preferably an anti-microtubule alkaloid agent.

Preferably, the cancer is resistant to a vinca alkaloid.

Preferably, the cancer is resistant to vincristine.

Preferably, the cancer is selected from neuroblastoma, breast cancer, oesophageal cancer or ovarian cancer.

Preferably, the neuroblastoma is metastatic neuroblastoma in the bone marrow.

Preferably, the neuroblastoma is vincristine-resistant metastatic neuroblastoma in the bone marrow.

Preferably, the breast cancer is invasive ductal carcinoma.

Preferably, the oesophageal cancer is oesophageal squamous cell carcinoma.

Preferably, the ovarian cancer is high grade ovarian serous adenocarcinoma or ovarian cystadenocarcinoma.

Preferably, the cancer is metastatic cancer.

Preferably, the subject is a mammal.

Preferably, the subject is a human.

Preferably, the compositions of the present invention comprise one or more additional active compounds. Preferably, the one or more additional active compounds are therapeutically active compounds, for example in the form of an additional therapeutic compound for co-delivery with the compositions described herein.

According to another aspect of the present invention, there is provided a method for producing a compound as described herein, wherein the method comprises extracting the compound from a plant of the genus Salix.

Preferably, the method comprises extracting the compound from leaf or stem tissue of a plant of the genus Salix.

Preferably, Salix is selected from (i) Salix miyabeana, Salix dasyclados, Salix gilgiana, Salix gmelinii, Salix repens, Salix capsica, Salix rehderiana, S. rossica, S. glaucophyloides or Salix adhenophylla, or (ii) a hybrid of Salix miyabeana, Salix dasyclados, Salix gilgiana, Salix gmelinii, Salix repens, Salix capsica, Salix rehderiana, S. rossica, S. glaucophyloides or Salix adhenophylla.

Preferably, Salix is selected from (i) Salix miyabeana, Salix dasyclados, Salix gilgiana, Salix gmelinii, Salix repens, Salix capsica or Salix adhenophylla, or (ii) a hybrid of Salix miyabeana, Salix dasyclados, Salix gilgiana, Salix gmelinii, Salix repens, Salix capsica or Salix adhenophylla.

Preferably, Salix is Salix miyabeana or a hybrid of Salix miyabeana.

Preferably, Salix is S. miyabeana Seemen or a hybrid of S. miyabeana Seemen.

Preferably, Salix is S. miyabeana purpurescens or a hybrid of S. miyabeana purpurescens.

Preferably, Salix is Salix dasyclados or a hybrid of Salix dasyclados.

Preferably, Salix is Salix rehderiana or a hybrid of Salix rehderiana.

Preferably, Salix is RRes 710-27, RR09102 hybrid [NWC607 S. rehderiana×RR05337 (Aud×S. rossica)].

Preferably, Salix is S. miyabeana hybrid breeding line (RR10347) generated from a cross of NWC941 (S. miyabeana purpurescens) with RR05326 (Resolution×S. rossica).

Preferably, Salix is willow breeding line RR10147.

RR10147 was developed as part of a biomass improvement programme at Rothamsted Research. This hybrid line included S. dasyclados (NWC577) in both parents [RR07187 (944 S. glaucophyloides×577 “77056”)×RR07188 (944 S. glaucophyloides×577 “77056”)] as well as S. glaucophyloides (NWC 944).

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention.

### DETAILED DESCRIPTION

Example embodiments of the present invention will now be described with reference to the accompanying Figures, in which:—

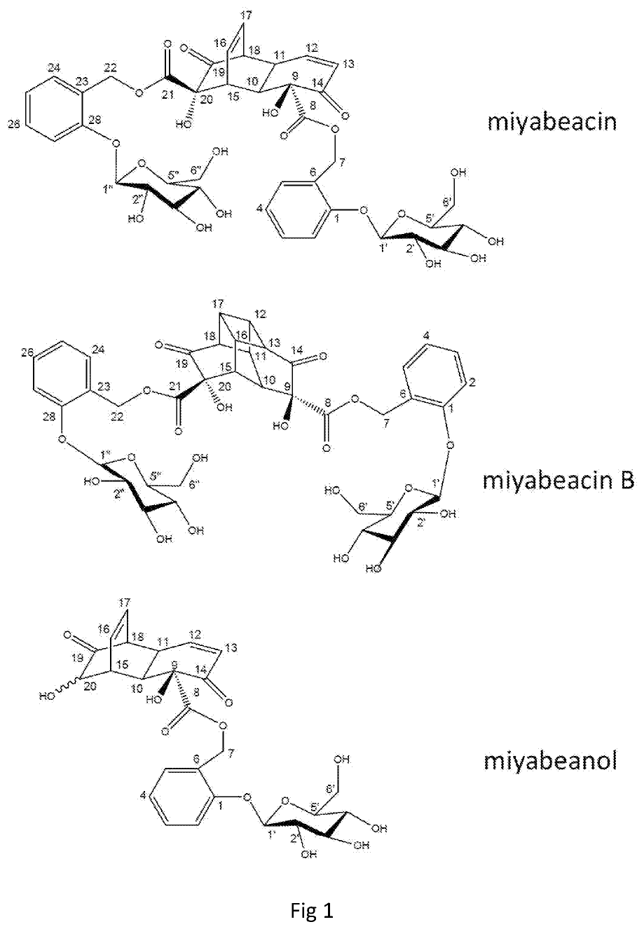

FIG. 1 shows structures of isolated dimeric compounds from Salix miyabeana;

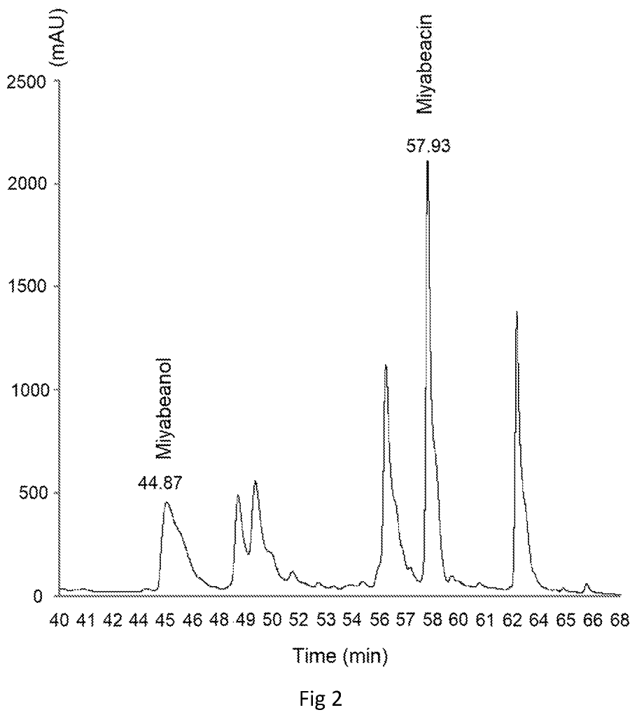

FIG. 2 shows reversed phase HPLC analysis of Salix miyabeana leaf extract indicating the peak of miyabeacin at 57.93 minutes;

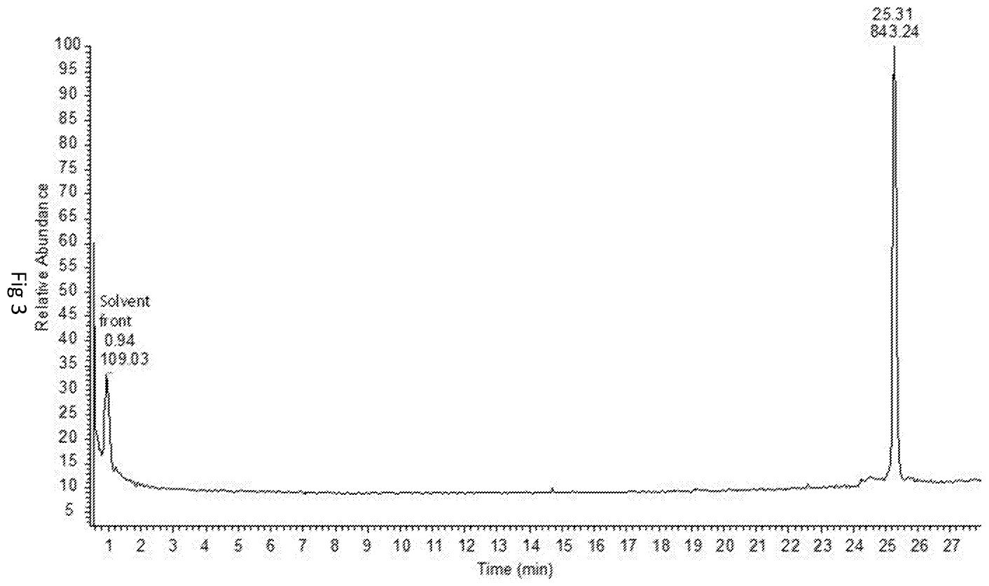

FIG. 3 shows Total Ion Chromatogram from LC-MS analysis (negative ion mode) of purified miyabeacin;

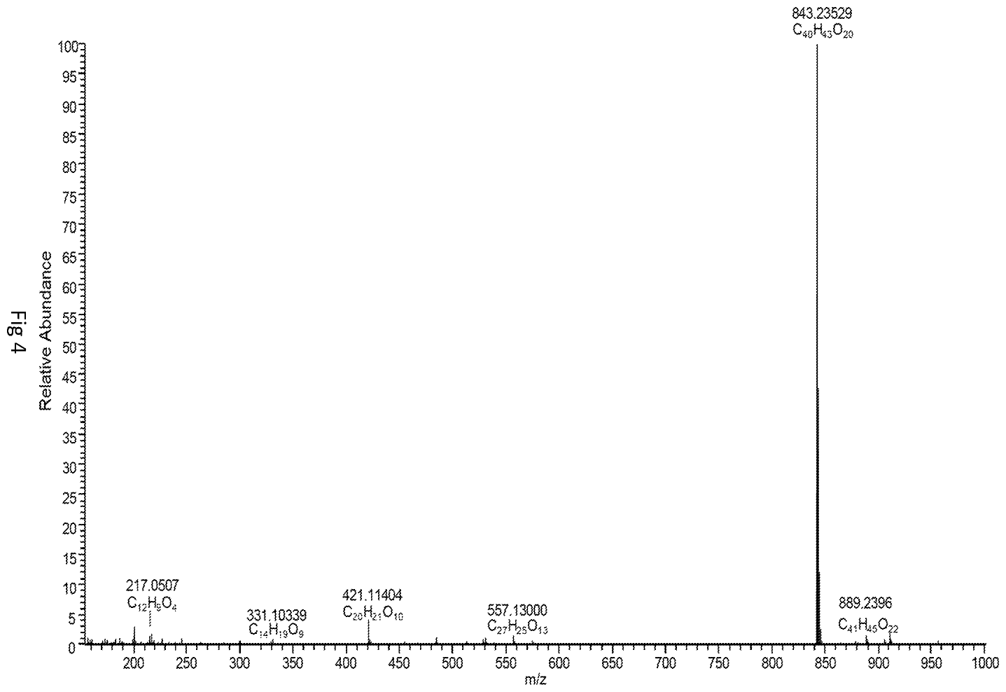

FIG. 4 shows mass spectrum (negative ion mode) of miyabeacin at m/z 843.23529 with retention time 25.31 min;

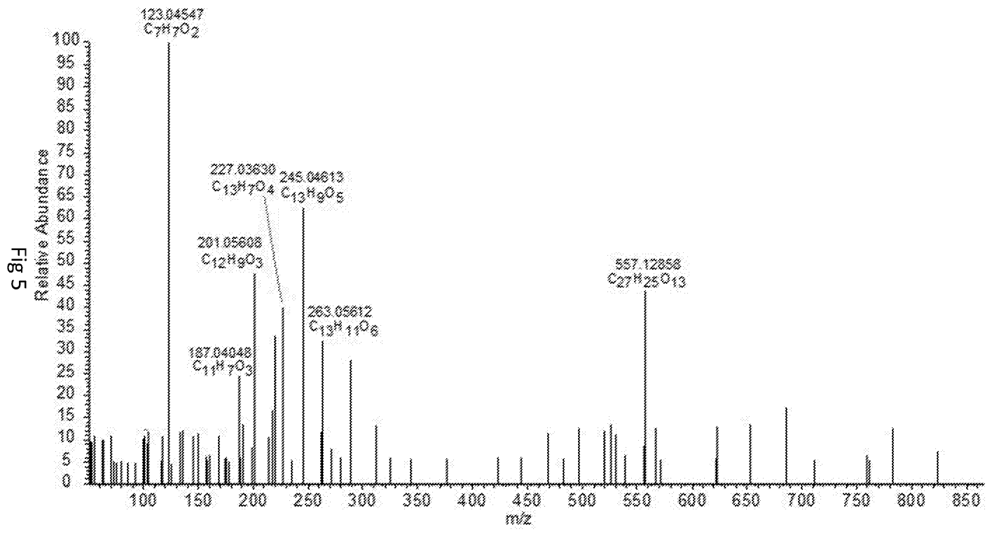

FIG. 5 shows MS-MS spectrum (negative ion mode) of m/z 843.23529 (25.31 min);

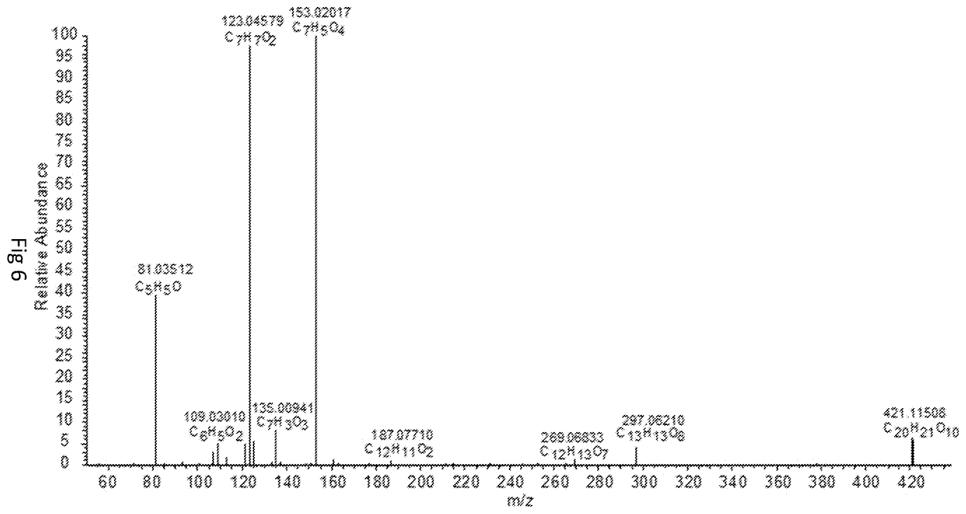

FIG. 6 shows MS-MS spectrum (negative ion mode) of m/z 421.11404 (25.31 min);

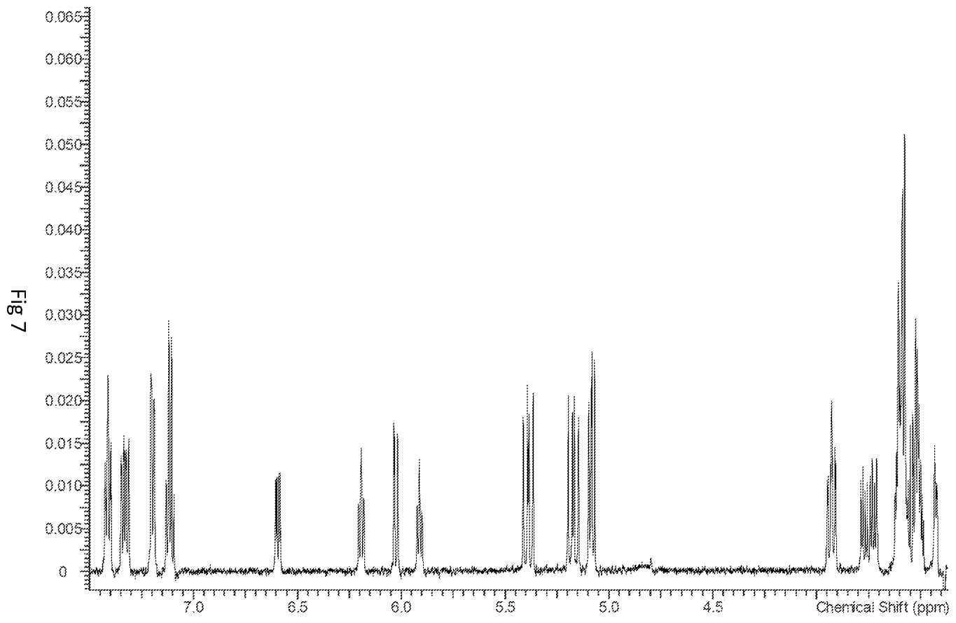

FIG. 7 shows ¹H-NMR spectrum of purified miyabeacin in CD3OD. Expanded region shown between δ7.50-δ3.49;

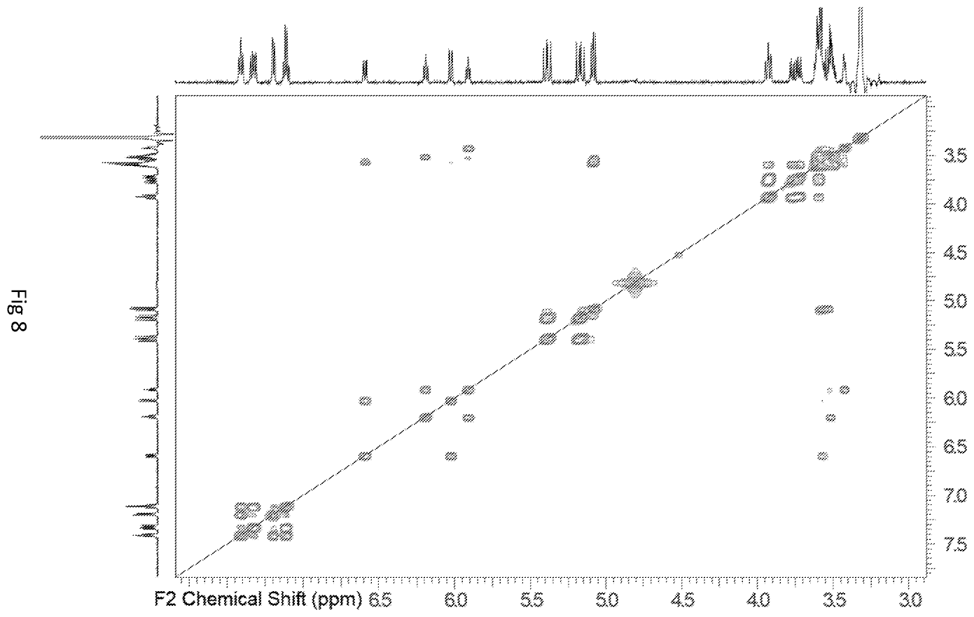

FIG. 8 shows ¹H-¹H COSY NMR spectrum of purified miyabeacin in CD₃OD. Expanded region shown between δ7.51-δ3.49;

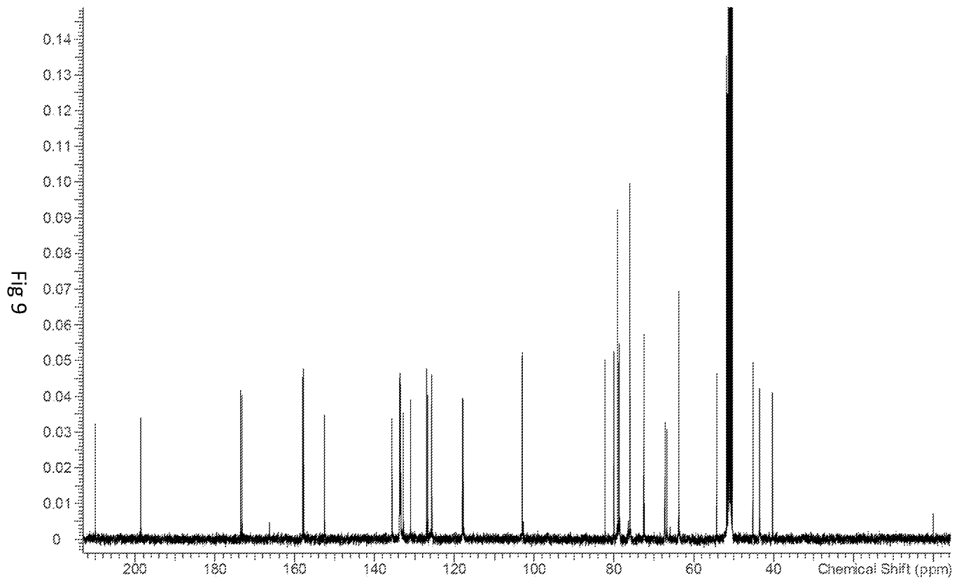

FIG. 9 shows ¹³C-NMR spectrum of purified miyabeacin in CD₃OD;

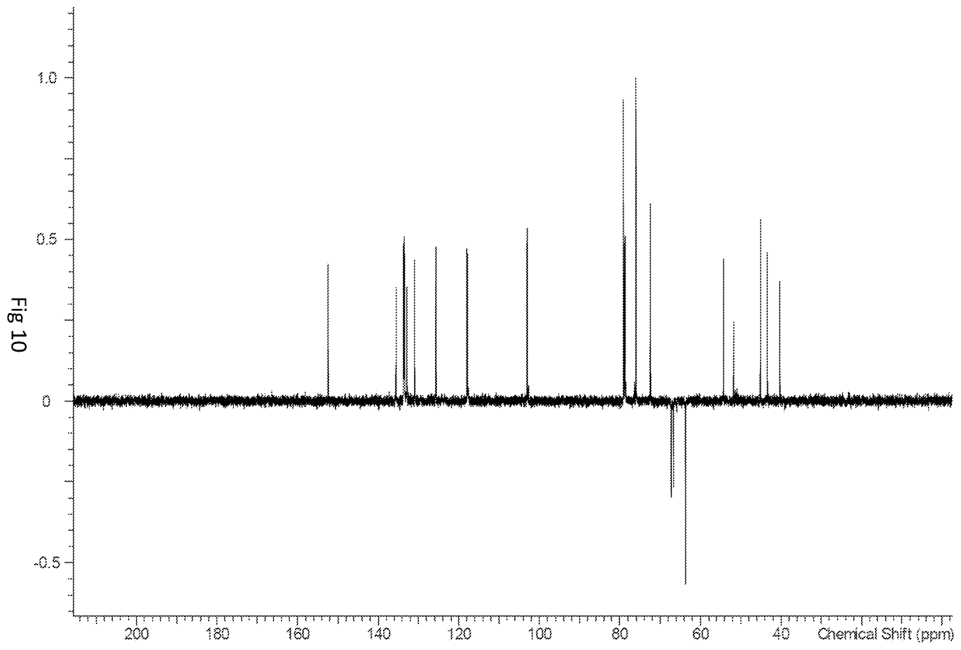

FIG. 10 shows ¹³C-DEPT135 spectrum of purified miyabeacin in CD₃OD;

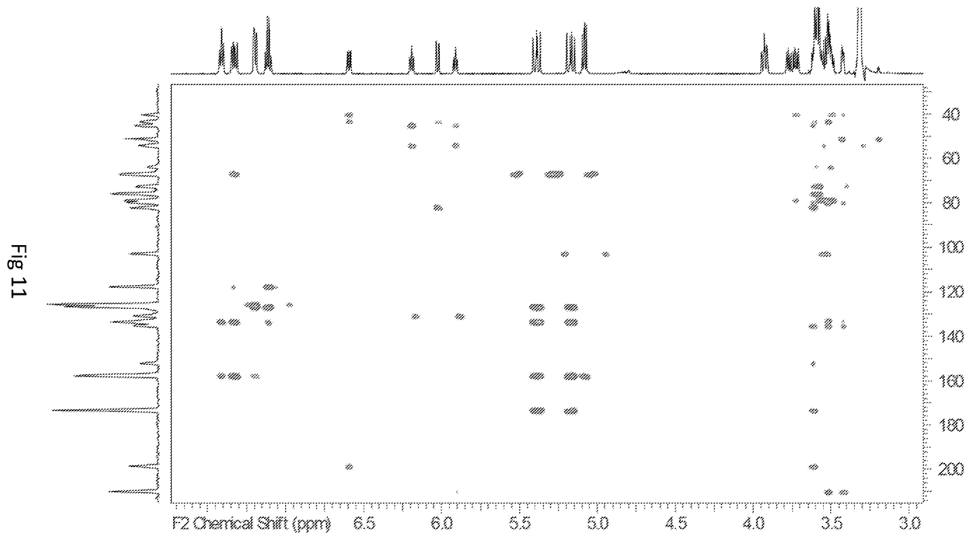

FIG. 11 shows ¹H-¹³C-HMBC spectrum of purified miyabeacin in CD₃OD;

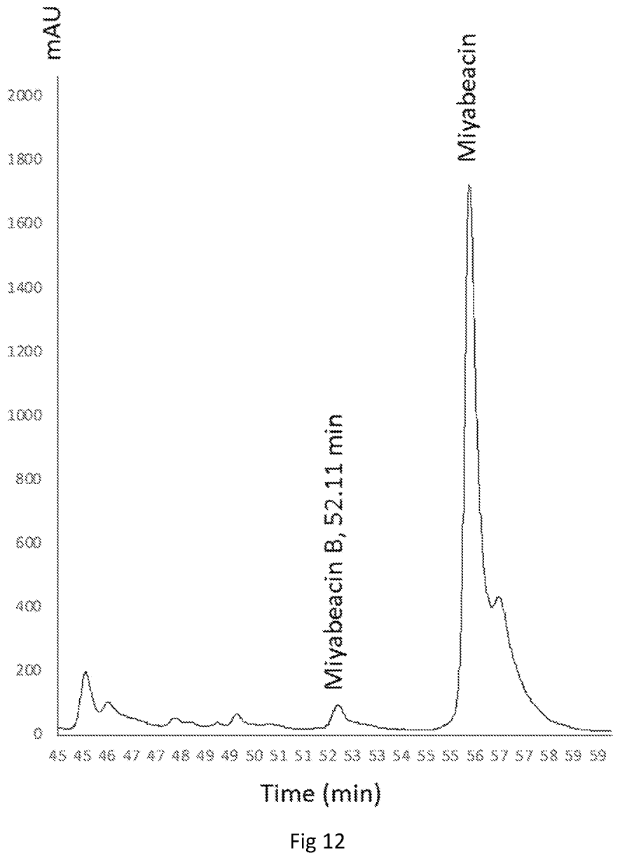

FIG. 12 shows reversed phase HPLC analysis of Salix miyabeana stem extract indicating the peak of miyabeacin B at 52.11 minutes;

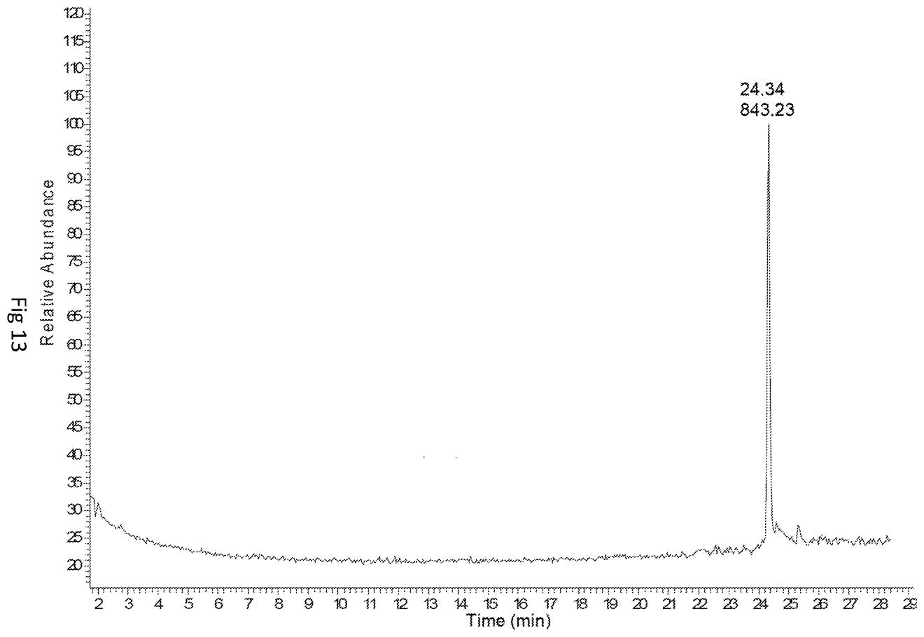

FIG. 13 shows Total Ion Chromatogram from LC-MS analysis (negative ion mode) of purified miyabeacin B;

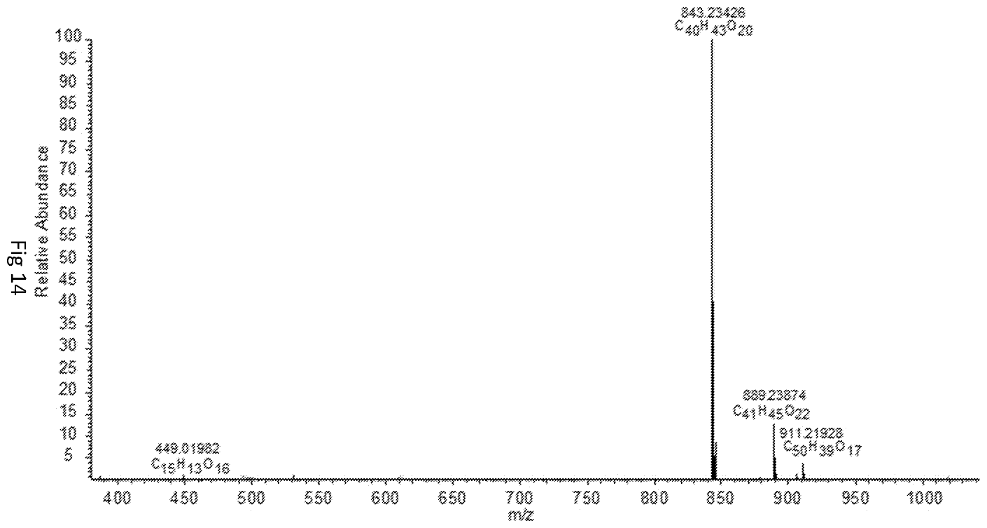

FIG. 14 shows mass spectrum (negative ion mode) of miyabeacin B at m/z 843.23474 with retention time 24.34 min;

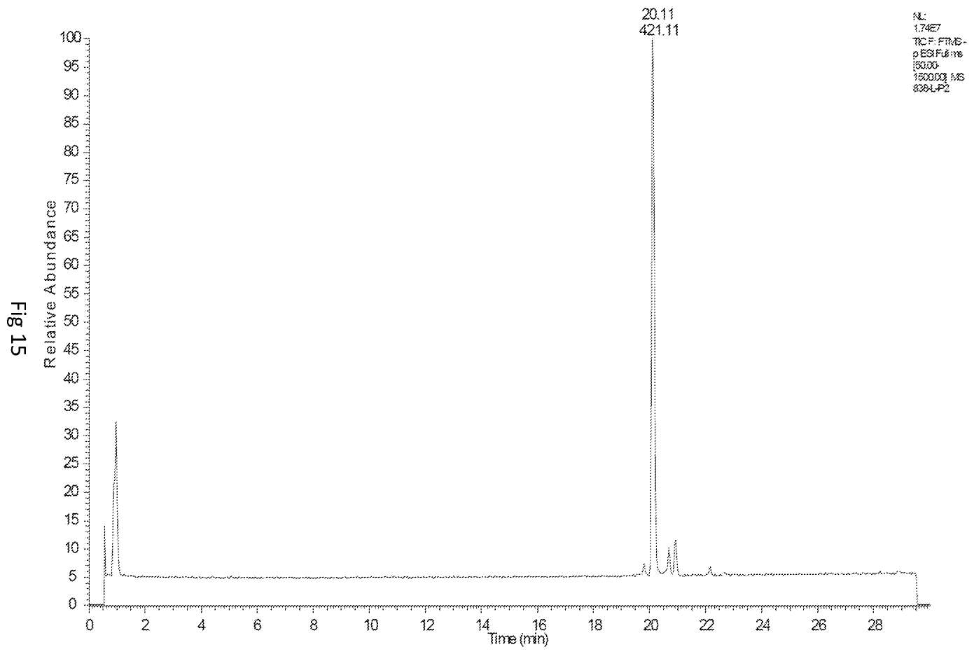

FIG. 15 shows Total Ion Chromatogram from LC-MS analysis (negative ion mode) of purified miyabeanol;

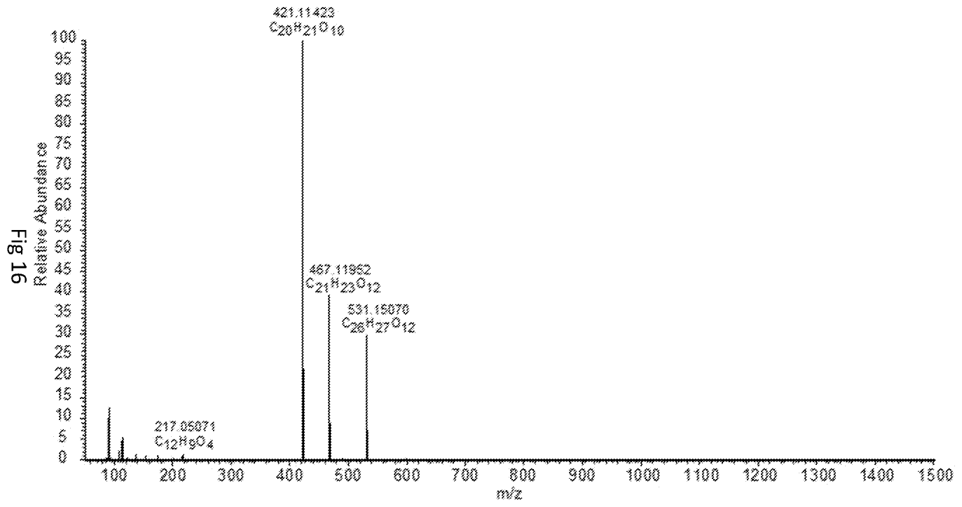

FIG. 16 shows mass spectrum (negative ion mode) of miyabeanol at m/z 531.15074 with retention time 20.11 min;

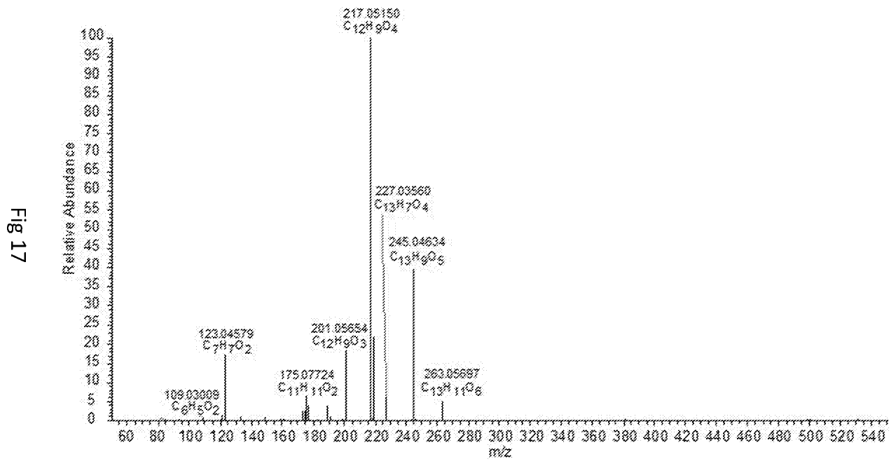

FIG. 17 shows MSMS spectrum (negative ion mode) of miyabeanol at m/z 531.15074 with retention time 20.11 min;

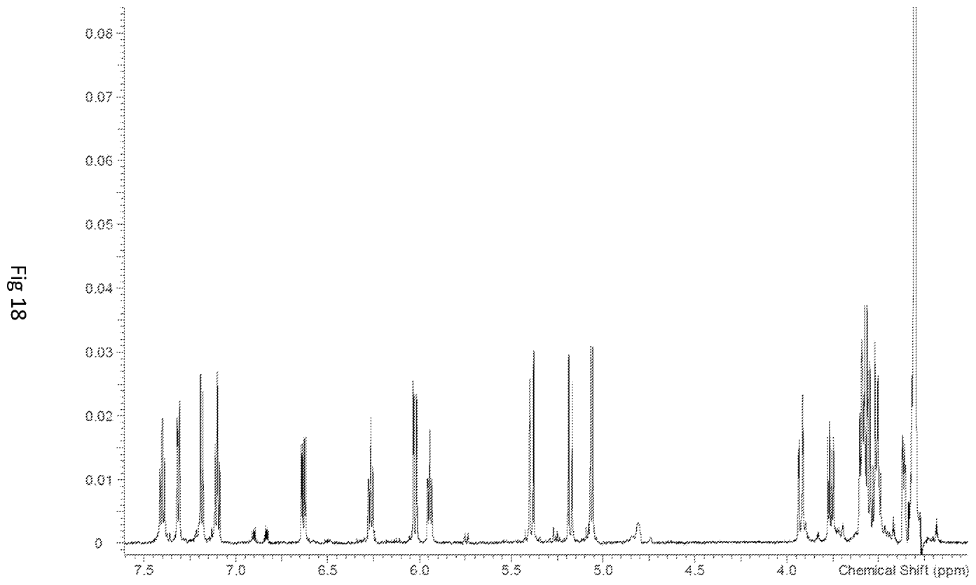

FIG. 18 shows ¹H-NMR spectrum of purified miyabeanol in D2O:CD3OD (4:1). Expanded region shown between δ7.60-δ3.00;

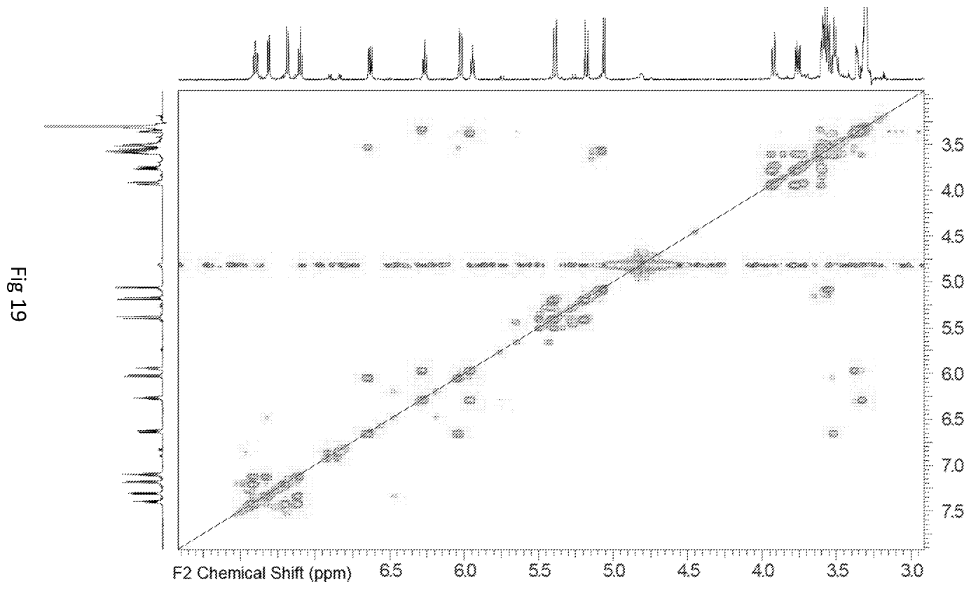

FIG. 19 shows ¹H-¹H COSY NMR spectrum of miyabeanol in D2O:CD3OD (4:1); and

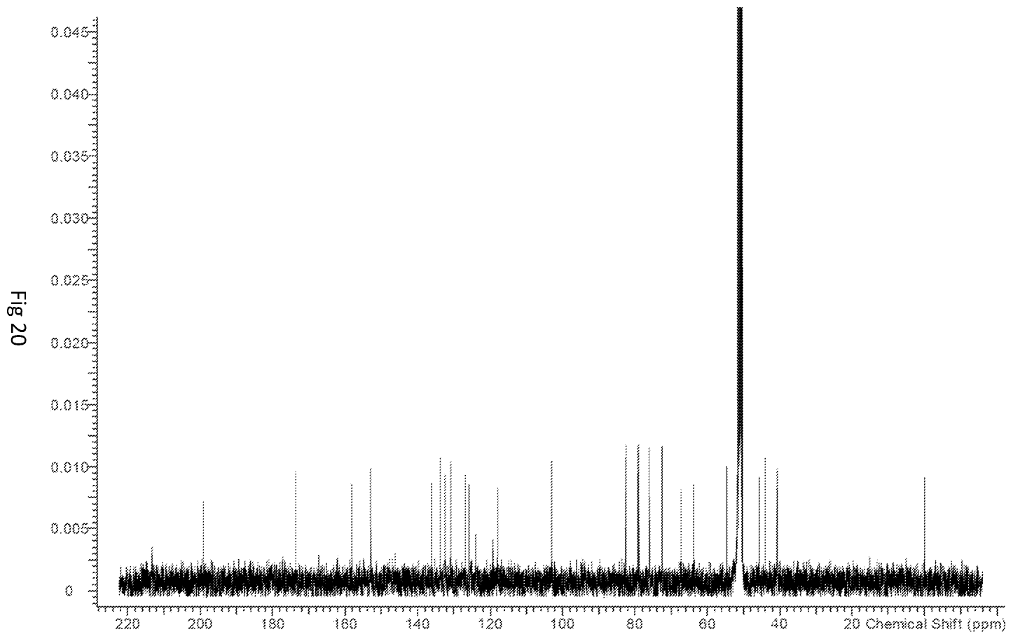

FIG. 20 shows ¹³C NMR spectrum of miyabeanol in D2O:CD3OD (4:1).

The present invention relates to novel compounds and their use in therapy, in particular for the treatment of cancer.

The compounds described herein were extracted from plants of the genus Salix, in particular Salix miyabeana or Salix dasyclados.

The genetic origin of Salix plants in general is unknown, although they are most abundant in cold and temperate regions of the Northern Hemisphere, including, for example Europe, Asia and North America.

In relation to Salix miyabeana referred to herein, this species is believed to be native to Japan and Korea.

In relation to Salix dasyclados referred to herein, this species is believed to be native to Siberia.

In relation to Salix gilgiana referred to herein, this species is believed to be native to Japan and Korea.

In relation to Salix gmelinii referred to herein, this species is believed to be native to Kazakhstan.

In relation to Salix repens referred to herein, this species is believed to be native to Austria, Baltic States, Belgium, Central European Russia, Czechoslovakia, Denmark, Finland, France, Germany, Great Britain, Ireland, Netherlands, North European Russia, Norway, Portugal, Spain, Sweden, Switzerland and Yugoslavia.

In relation to Salix capsica referred to herein, this species is believed to be native to Central Asia.

In relation to Salix adhenophylla referred to herein, this species is believed to be native to North America.

In relation to Salix rehderiana referred to herein, this species is believed to be native to China.

In relation to Salix rossica referred to herein, this species is believed to be native to Europe, Western Asia, and the Himalayas.

In relation to Salix glaucophyloides referred to herein, this species is believed to be native to North America.

Within this specification, the term “miyabeacin” means a compound of Formula X.

Within this specification, the term “miyabeacin B” means a compound of Formula XI.

Within this specification, the term “miyabeanol” means a compound of Formula XII.

Within this specification, the term “about” means plus or minus 20%, more preferably plus or minus 10%, even more preferably plus or minus 5%, most preferably plus or minus 2%.

As used herein, the term “therapeutically effective amount” means the amount of a composition which is required to reduce the severity of and/or ameliorate at least one condition or symptom which results from the disease in question.

Within this specification, the term “treatment” means treatment of an existing disease and/or prophylactic treatment in order to prevent incidence of a disease. As such, the methods and compositions of the invention can be used for the treatment, prevention, inhibition of progression or delay in the onset of disease.

Within this specification, reference to a “a compound as described herein” preferably means a compound of any of Formulas I to XX, or a derivative, homologue, stereoisomer, prodrug or pharmaceutical salt thereof.

Within this specification, reference to “a composition as described herein” means a composition comprising a compound as described herein. Preferably, the composition is a pharmaceutical composition.

Preferably, the composition comprises a therapeutically effective amount of at least one compound as described herein or a physiologically tolerated salt thereof.

Preferably, the composition comprises a physiologically tolerated carrier.

Within this specification, the term “prodrug” means to a compound that is biologically inactive, but is metabolized to produce an active therapeutic drug.

Within this specification, the term “derivative” means a molecule derived from the compounds described herein. Such derivatives may, for example, be synthetically altered derivatives of these compounds.

Within this specification, the term “homologue” refers to a molecule having substantial structural similarities to the compounds described herein.

Within this specification, the term “stereoisomer” means a molecule that has the same molecular formula and sequence of bonded atoms as another molecule, but which differs in the three-dimensional orientations of its atoms in space.

The compounds and compositions of the present invention can be formulated for clinical use into pharmaceutical formulations for administration by any suitable route. Examples include via oral, nasal, rectal, topical, sublingual, transdermal, intrathecal, transmucosal or parenteral (e.g. subcutaneous, intramuscular, intravenous and intradermal) administration.

Pharmaceutical formulations can be prepared by mixing the active substance, or a pharmaceutically acceptable salt thereof, with conventional pharmaceutically acceptable carriers, diluents or excipients. Examples of excipients include water, gelatin, gum arabicum, lactose, microcrystalline cellulose, starch, sodium starch glycolate, calcium hydrogen phosphate, magnesium stearate, talcum, colloidal silicon dioxide, and so on. Pharmaceutically acceptable carriers include solvents, dispersion media, coatings, antibacterial and antifungal agents, isotonic and absorption delaying agents, and the like, compatible with pharmaceutical administration, unless use thereof is incompatible with the active compound.

The formulations may also contain other pharmacologically active agents, and conventional additives, such as stabilizers, wetting agents, emulsifiers, flavouring agents, buffers, and the like.

The formulations may be prepared by conventional methods in dosage forms such as tablets, capsules, granules, powders, syrups, suspensions, suppositories or injections. Liquid formulations may be prepared by dissolving or suspending the active substance in water or other suitable vehicles. Tablets and granules may be coated in a conventional manner.

Solutions or suspensions used for parenteral, intradermal, or subcutaneous application can include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

For oral administration, the compositions can be in the form of soft gelatin capsules or tablets and will usually include an inert diluent or an edible carrier. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

Formulations intended for inhalation can be provided as an aerosol spray, for example in a pressurised container or dispenser which contains a suitable propellant.

Transmucosal or transdermal delivery means can be used for systemic administration. Penetrants appropriate to the barrier in question can be used and are well known in the art. Examples include detergents, bile salts, and fusidic acid derivatives. Nasal sprays and suppositories can be used for transmucosal delivery. Creams, ointments, salves and gels can be used for transdermal delivery. In the case of rectal delivery, the formulations can also be provided as retention enemas.

Formulations intended for targeted delivery of the compositions and compounds described herein can also be provided, for example using targeting agents such as antibodies, antibody fragments, receptor binding agents, nanoparticles, nanocarriers or combinations thereof. In this respect, it is known that cancer cells exhibit cancer specific markers which means that agents specific for these markers can be used to direct the compounds and compositions described herein to cancer cells and tissues in a selective manner.

In one example, the compounds described herein can be bound to antibodies or fragments thereof specific for one or more cancer cell specific markers or conjugated to nanoparticles coupled to a targeting ligand specific for one or more cancer cell specific markers. Examples of nanoparticles include lipid cationic nanoparticles, gold nanoparticles, silica nanoparticles, PEGylated nanoparticles and amphiphilic polymeric nanoparticles. The compositions can comprise nanoparticles with multiple functional ligands which can include, for example, diagnostic and/or other therapeutic agents in addition to the compounds described herein.

Nanocarriers, such as liposomes and micelles, conjugated to targeting molecules, such as ligands, antibodies or antibody fragments, can be used to deliver unmodified compounds and compositions described herein to cancer cells and tissues.

The compounds and compositions may also be provided in formulations which prevent rapid elimination from the body. Examples include known modified release formulations such as implants and microencapsulated delivery systems.

Pharmaceutical compositions containing the appropriately formulated compound can be included in a container, pack, or dispenser together with instructions for administration.

The appropriate dosage form for the formulation will depend upon the intended route of administration, the required quantity of drug to be delivered and the potential toxicity of the compound. This can be determined in accordance with standard procedures known in the art.

For example, toxicity and therapeutic efficacy of compounds can be determined by standard pharmaceutical procedures in cell cultures or experimental animals and evaluated by considering the LD50 (the dose lethal to 50% of the population), the ED50 (the dose therapeutically effective in 50% of the population) and the resultant therapeutic index (LD50/ED50). Appropriate dosage forms may also depend upon the potential side effects of particular routes of delivery and the amount of active compound required to effect sufficient delivery to the intended site of therapeutic need.

### EXAMPLES

### Isolation and Characterisation of Dimeric Compounds Miyabeacin, Miyabeacin B and Miyabeanol.

Freeze dried juvenile leaves of Salix miyabeana were used as the starting material for the initial isolation of Miyabeacin (FIG. 1A) and Miyabeanol (FIG. 1C). Freeze dried juvenile stems of Salix miyabeana were used as the starting material for the initial isolation of Miyabeacin B (FIG. 1i). Tissue was milled to a homogeneous powder prior to extraction.

### Miyabeacin

For the initial isolation of Miyabeacin, 1 mL of water:methanol (80:20) was added to Salix miyabeana leaf tissue (50 mg). The suspension was agitated for 5 minutes at room temperature and then heated to 50° C. for 10 minutes using a water bath. The resultant solution was centrifuged at 13,000 rpm for 5 minutes. 800 μL of the supernatant, removed to a clean tube, was heated at 90° C. for 2 minutes. The solution was cooled (5° C.) for 30 minutes and centrifuged at 13,000 rpm for 5 minutes. The supernatant, containing the target compound was subjected to purification using reversed-phase HPLC. 6 repeated injections of 100 μL each were made into an analytical HPLC using an Agilent 1100 HPLC system equipped with a quaternary pump, diode array detector, column oven and auto sampler. Peaks were separated using an Ascentis C18 column (Sum, 5×250 mm (Supelco, UK). The operating solvents were: Solvent A: H2O with 0.1% formic acid, solvent B: acetonitrile with 0.1% formic acid. The operating gradient for peak isolation was from 5% B (0-10 min), 22% B (10-50 min) to 37% B (60-70 min) at a constant flow of 1 mL/min and using a total chromatographic run of 72 min. Peaks were identified and monitored using a wavelength of 254 nm and were collected manually into glass tubes. Miyabeacin eluted at 57.93 min (FIG. 2). Equivalent fractions from 6 runs were combined and evaporated using a Speedvac concentrator (Genevac, Suffolk, UK) to yield 1.68 mg of purified Miyabeacin. It was also possible to recover the product from the following Salix species by the procedures described above: Salix dasyclados, Salix gilgiana, Salix gmelinii, Salix repens, Salix capsica and Salix adhenophylla and hybrids thereof.

TABLE 2
Extraction and HPLC gradient conditions for the isolation of dimeric metabolites.

| | | | | | HPLC | | |
|---|---|---|---|---|---|---|---|
| | | | | | Gradient | | |
| | | | | | [mobile | | |
| | | | | | phases | | |
| | | | | | water (A) | | |
| | | | | | and | | |
| | | | | | acetonitrile | | |
| | | | | | (B), | | |
| | | | | Number | both | | |
| | | | Extraction | of 100 μL | containing | HPLC | |
| | | | Volume | Injections | 0.1% | Retention | |
| | Amount | | (solvent: | made into | formic | time of | Amount |
| Compound | Extracted | Tissue | H₂O:MeOH) | HPLC | acid.] | Peak | Isolated |
| X | 50 mg | Salix | 1 mL | 6 | 5% B | 57.93 | 1.68 |
| | | miyabeana | | | (10-0 min), | min | mg |
| | | Seemen. III | | | 22% B | | |
| | | leaf tissue. | | | (10-50 | | |
| | | Line: | | | min) to 37% | | |
| | | NWC837 | | | B (60- | | |
| | | | | | 70 min). | | |
| XI | 200 mg | Salix | 2.5 mL | >10 | 5% B | 52.11 | 0.67 |
| | | miyabeana | | | (0-10 min), | min | mg |
| | | Seemen. | | | 29% B | | |
| | | “Purpurescens” | | | (10-60 | | |
| | | stem tissue. | | | min) to 29% | | |
| | | Line: NWC941 | | | B (60- | | |
| | | | | | 70 min) | | |
| XII | 150 mg | Salix | 2 mL | 8 | 5% B | 44.87 | 1.05 |
| | | miyabeana | | | (0-10 min), | min | mg |
| | | Seemen. III | | | 22% B | | |
| | | leaf tissue. | | | (10-50 | | |
| | | Line: | | | min) to 37% | | |
| | | NWC837 | | | B (60- | | |
| | | | | | 70 min) | | |
| XIX | 450 mg | Salix | 4.5 mL | 44 | 20% B | 20.9 | 0.9 |
| | | miyabeana | | | (0-20 min), | min | mg |
| | | Seemen. | | | 40% B | | |
| | | “Purpurescens” | | | (20-25 | | |
| | | leaf tissue. | | | min) to 50% | | |
| | | Line: NWC941 | | | B (25- | | |
| | | | | | 35 min) | | |
| XIII & | 150 mg | RRes 710-27, | 2.4 mL (2 | 10 | 20% B | 41.4 | 0.75 |
| XIV | (2 × 75 | RR09102 | 1.2 mL) | | (0 min), 40% | | mg |
| | mg) | hybrid | | | B (0-45 | | |
| | | [NWC607 S. | | | min) to | | |
| | | rehderiana × | | | 100% B | | |
| | | RR05337 | | | (45.0-50 | | |
| | | (Aud × S. | | | min) | | |
| | | rossica)] leaf | | | | | |
| | | tissue | | | | | |
| XV | 150 mg | RRes 710-27, | 2.4 mL (2 | 10 | 20% B | 45.5 | 0.25 |
| | (2 × 75 | RR09102 | 1.2 mL) | | (0 min), 40% | | mg |
| | mg) | hybrid | | | B (0-45 | | |
| | | [NWC607 S. | | | min) to | | |
| | | rehderiana × | | | 100% B | | |
| | | RR05337 | | | (45.0-50 | | |
| | | (Aud × S. | | | min) | | |
| | | rossica)] leaf | | | | | |
| | | tissue | | | | | |

A further example, extending the range of substituted dimeric compounds was seen in the LC-MS analysis of a willow breeding line (RR10147) developed as part of a biomass improvement programme at Rothamsted Research. This hybrid line included S. dasyclados (NWC577) in both parents [RR07187 (944 S. glaucophyloides×577 “77056”)×RR07188 (944 S. glaucophyloides×577 “77056”)] as well as S. glaucophyloides (NWC 944). In the Total Ion Chromatogram of the negative ion mode LC-MS data salicortin, 2′-O-acetylsalicortin and tremulacin appeared as major peaks. Given that this cross has generated a hybrid capable of producing both acetylated and benzoylated salicinoids alongside salicortin it followed that associated dimeric analogues would also be expected to be formed via a matrix of cross-over reactions involving the three corresponding dienones. This was indeed the case with miyabeacin appearing at 25.03 min, 2′/2″-O-acetylmiyabeacin (Formula XIII/XIV) appearing at 26.90 min and 2′/2″-O-benzoylmiyabeacin (Formula XVI/XVII) appearing at 30.95 min. A further intriguing peak was observed at 32.48 min which showed an ion at m z 989.2617, corresponding to a formula of C₄₉H₄₉O₂₂. Although there was insufficient for isolation, the MS was suggestive of the predicted miyabeacin analogue bearing both an acetyl and benzoyl substitution.

The structure of Miyabeacin was determined by various forms of spectroscopy. Table 3 shows the general measurement conditions for spectroscopic analyses.

TABLE 3
General Conditions and parameters for spectroscopic measurements.
Measurement Conditions

| High resolution LC-MS | |
|---|---|
| LC apparatus | Ultimate 3000 RS uHPLC (Thermo) |
| Chromatography Column | C₁₈ Hypersil gold column |
| | (1.9 μm, 30 × 2.1 mm i.d.) |
| Column Temperature | 35° C. |
| Solvents | Water/0.1% formic acid (A) and |
| | acetonitrile/0.1% formic acid (B) |
| Solvent Gradient | 0 min, 0% B; 27 min, 70% B; |
| | 28 min, 100% B. |
| Flow rate | 0.3 mL/min |
| Run time | 30 min |
| Injection volume | 10 μL |
| MS Apparatus | LTQ-Orbitrap Elite (Thermo) |
| Source | Heated ESI source |
| Ionisation mode | Negative |
| Resolution | 120,000 |
| Capillary temperature | 350° C. |
| Source heater temperature | 350° C. |
| Source voltage | 2500 V |
| Source current | 100 uA |
| Sheath gas flow | 35 |
| Auxiliary gas | 10 |
| R.F. Lens | 50% |
| Scan range | m/z 50-1500 |
| MS-MS fragmentation | Automatic on top 3 ions |
| Ion isolation width for MSMS | m/z 2 |
| Fragmentation mode | HCD |
| Normalised collision energy | 65 |
| Activation time | 0.1 ms |
| NMR | |
| Apparatus | Avance 600 (Bruker) |
| Observation Frequency | ¹H: 600.05, ¹³C: 150.9 |
| Solvent | D₂O:CD₃OD (80:20) |
| Concentration | 0.6 mg/mL |
| Internal Standard | d₄-TSP |
| Temperature | 300 K |
| Probe | 5 mm Selective Inverse |
| ¹H NMR Measurement | |
| Pulse sequence | zgpr |
| Sweep width | 7183 Hz |
| Spectrum offset | 2879.40 Hz |
| Data points | 32,768 |
| Pulse angle | 90° |
| Delay | 5 s |
| Number of scans | 64 |
| 2D COSY 45 Measurement | |
| Pulse program | cosyqf45 |
| Observation width | 2973, 2973 Hz |
| Data points | 1024, 1024 |
| Temperature | 300 K |
| Number of transients | 32 |
| 2D HSQC Measurement | |
| Pulse program | hsqcetgpsi2 |
| Observation width | 7180, 30150 Hz |
| Data points | 2048, 1024 |
| Temperature | 300 K |
| Number of transients | 128 |
| 2D HMBC Measurement | |
| Pulse program | hmbcgpndqf |
| Observation width | 7182, 33165 Hz |
| Data points | 4096, 256 |
| Temperature | 300 K |
| Number of transients | 256 |
| ¹³C NMR | |
| Apparatus | Avance 400 (Bruker) |
| Observation Frequency | ¹³C: 100.61 |
| Solvent | D₂O:CD₃OD (80:20) |
| Concentration | 0.6 mg/mL |
| Internal Standard | d₄-TSP |
| Temperature | 300 K |
| Probe | 5 mm Broadband BBO |
| ¹³C NMR Measurement | |
| Pulse sequence | dept135 |
| Sweep width | 23,980 Hz |
| Spectrum offset | 10363 Hz |
| Data points | 32768 |
| Pulse angle | 30° |
| Delay | 0.7 s |
| Number of scans | 46,191 |
| DEPT Measurement | |
| Observation width | 23980 Hz |
| Data points | 65536 |
| Pulse repetition time | 2 |
| Number of scans | 4096 |

Abbreviations
DEPT: Distortionless Enhancement by Polarization Transfer (A method for determining a carbon type (distinguishing among CH3, CH2, CH, and C))
COSY: Correlation SpectroscopY (A method of ¹H-¹H COSY)
HSQC: Heteronuclear Single Quantum Coherence (A method of ¹H-¹³C COSY)
HMBC: Heteronuclear Multiple Bond Correlation (A method of long-range ¹H-¹³C COSY)

### Miyabeacin Spectroscopic Analyses

High Resolution LC-MS: LC-MS was carried out in negative ion mode using a C18 column. Conditions of analysis are outlined in Table 3. FIG. 3 shows a total ion chromatogram of purified miyabeacin which appeared as a single peak at 25.31 min. A high-resolution mass spectrum (FIG. 4) was collected in negative ion mode and showed an m/z ion at 843.23529 (C₄₀H₄₃O₂₀), corresponding to the [M-H]− of miyabeacin (molecular formula C₄₀H₄₄O₂₀). Smaller ions also present in the mass spectrum were m/z 889.2396 (C₄₁H₄₅O₂₂, formate adduct), 557.1300 (C₂₇H₂₅O₁₃), 421.1140 (C₂₀H₂₁O₁₀), 331.1034 (C₁₄H₁₉O₉) and 217.0507 (C₁₂H₉O₄). MS-MS of m/z 843.23529 (FIG. 5) revealed a variety of low abundance fragments including m/z 123.04538 (C₇H₇O₂), 201.05629 (C₁₂H₉O₃), 227.03554 (C₁₃H₇O₄), 245.04494 (C₁₃H₉O₅) and 557.13739 (C₂₇H₂₅O₁₃). MS-MS of m/z 421.11404 ion (FIG. 6) gave fragments at m/z 297.06246 (C₁₃H₁₃O₈), 153.02017 (C₇H₅O₄), 135.00946 (C₇H₃O₃), 123.04583 (C₇H₇O₂), 109.03004 (C₆H₅O₂) and 81.03513 (C₅H₅O).

NMR spectroscopy: ¹H-NMR data of miyabeacin was collected at 600 MHz in aqueous d₄-methanol containing 0.01% w/v d₄-TSP as internal standard. The spectrum showed peaks relating to 34 coupled protons (FIG. 7 and Table 4).

TABLE 4
¹H-NMR assignments for miyabeacin. Data collected at 600
MHz in D₂O:CD₃OD (4:1), referenced to d₄-TSP (0.01% w/v).

| Position | | |
|---|---|---|
| 1 | — | |
| 2 | 7.19 | (d, 8.3) |
| 3 | 7.41 | (ddd, 8.0, 7.5, 2.0) |
| 4 | 7.12 (t, 7.5)/7.11 (t, 7.5) | |
| 5 | 7.32 (dd, 7.6, 1.5)/7.34 (dd, 7.6.1.5) | |
| 6 | — | |
| 7α | 5.40 | (d, 11.9) |
| 7β | 5.19 | (d, 11.9) |
| 8 | — | |
| 9 | — | |
| 10 | 3.59-3.63 | (m) |
| 11 | 3.58-3.55 | (m) |
| 12 | 6.59 | (dd, 10.2, 4.1) |
| 13 | 6.02 | (dd, 10.2, 1.5) |
| 14 | — | |
| 15 | 3.50-3.53 | (m) |
| 16 | 6.19 | (t, 7.9) |
| 17 | 5.91 | (ddd, 7.9, 6.5, 1.4) |
| 18 | 3.43 | (m) |
| 19 | — | |
| 20 | — | |
| 21 | — | |
| 22β | 5.38 | (d, 12.1) |
| 22α | 5.16 | (d, 12.1) |
| 23 | — | |
| 24 | 7.32 (dd, 7.6, 1.5)/7.34 (dd, 7.6, 1.5) | |
| 25 | 7.12 (t, 7.5)/7.11 (t, 7.5) | |
| 26 | 7.41 | (ddd, 8.0, 7.5, 2.0) |
| 27 | 7.20 | (d, 8.3) |
| 28 | — | |
| 1′ | 5.09 (d, 7.5)/5.07 (d, 7.8) | |
| 2′ | 3.55-3.63 | (m) |
| 3′ | 3.56-3.62 | (m) |
| 4′ | 3.47-3.52 | (m) |
| 5′ | 3.56-3.62 | (m) |
| 6′β | 3.77 (dd, 12.4, 6.0)/3.73 (dd, 12.4, 6.0) | |
| 6′α | 3.94 (dd, 12.4, 2.1)/3.92 (dd, 12.4, 2-1) | |
| 1″ | 5.09 (d, 7.5)/5.07 (d, 7.8) | |
| 2″ | 3.55-3.63 | (m) |
| 3″ | 3.56-3.62 | (m) |
| 4″ | 3.47-3.52 | (m) |
| 5″ | 3.56-3.62 | (m) |

| | |
|---|---|
| 6″β | 3.77 (dd, 12.4, 6.0)/3.73 (dd, 12.4, 6.0) |
| 6″α | 3.94 (dd, 12.4, 2.l)/3.92 (dd, 12.4, 2.1) |

Four signals were observed between δ 7.34-7.10 and were consistent with those obtained in salicyl containing compounds. Integration of these aromatic peaks corresponded to 8 protons suggestive of two such salicyl rings. This was confirmed by the presence of two pairs of J=12 Hz doublet signals relating to the distinctive salicyl hydroxymethylene group (pair 1: δ5.40 and δ5.19; pair 2: δ5.38 and δ5.16). Similarly, the molecule contained two separate glucoside moieties with characteristic doublet signals relating to the H-1′ anomeric protons being duplicated (δ5.09 and δ5.07) as were those corresponding to the glucosyl 6′-methylenes. Four separate olefin signals were present between δ6.60 and 5.85 each integrating for one proton, two appearing as double doublets and the others as simple triplets. ¹H-¹H COSY analysis (FIG. 8) demonstrated that the two double bonds were isolated from each other. Integration of the carbohydrate region (δ3.96-3.40) suggested a total of 16 coupled protons. Of these, 12 could be accounted for in two glucose units leaving 4 unaccounted for. ¹³C NMR data (FIG. 9 and Table 5) confirmed the presence of 40 carbon atoms in the molecule including two ketone carbonyls at δ199.6 and 210.0 and two ester carbonyls at δ 173.6 and 173.2 while ¹³C DEPT135 (FIG. 10) identified four non-aromatic methine signals, in addition to those of glucose (×2) and two olefinic signals.

TABLE 5
¹³C-NMR assignment for mivabeacin. Data collected at 100.61
MHz in D₂O:CD₃OD (4:1), referenced to d₄-TSP (0.01% w/v).

| Position | |
|---|---|
| 1 | 158.0 |
| 2 | 117.9 |
| 3 | 133.5/133.4 |
| 4 | 125.6/125.7 |
| 5 | 133.7/133.6 |
| 6 | 126.9 |
| 7α | 67.3 |
| 7β | 67.3 |
| 8 | 173.6 |
| 9 | 82.2 |
| 10 | 40.3 |
| 11 | 43.5 |
| 12 | 152.5 |
| 13 | 130.9 |
| 14 | 198.6 |
| 15 | 45.1 |
| 16 | 135.5 |
| 17 | 132.8 |
| 18 | 54.2 |
| 19 | 210.0 |
| 20 | 80.0 |
| 21 | 173.2 |
| 22β | 66.7 |
| 22α | 66.7 |
| 23 | 126.6 |
| 24 | 133.7/133.6 |
| 25 | 125.6/125.7 |
| 26 | 133.5/133.4 |
| 27 | 117.7 |
| 28 | 157.7 |
| 1′ | 103.0/102.9 |
| 2′ | 76.0 |
| 3′ | 79.1 |
| 4′ | 72.5/72.4 |
| 5′ | 78.7/78.8 |
| 6′β | 63.7 |
| 6′α | 63.7 |
| 1″ | 103.0/102.9 |
| 2″ | 76.0 |
| 3″ | 79.1 |
| 4″ | 72.5/72.4 |
| 5″ | 78.7/78.8 |
| 6″β | 63.7 |
| 6″α | 63.7 |

Given the molecular formula from accurate mass, the similarity in fragmentation pattern of the smaller m/z 421 fragment to that of the known molecule salicortin, and the duplication of benzyl and glycosyl related NMR signals we postulated that miyabeacin was an unsymmetrical dimeric structure formed via conjugation of two molecules of a dehydro analogue of salicortin. The structure has a tricyclododecadiene core. Key correlations in the ¹H-¹³C HMBC were observed around all the positions of the dimeric core structure (FIG. 11) and between H-10 (δ 3.60) and C-8 (δ 173.6) confirming the attachment of a carboxyl group to the core structure at C-9. The correlation between H-7 (δ 5.19 and 5.40) and C-8 (δ 173.6) confirmed the linkage via the ester carbonyl, to a salicyl moiety. Similar correlations were observed between H-15 (δ 3.50) to C-21 (δ 173.2) and also between H-22 (δ 5.16 and 5.38) and C-21 (δ 173.2), suggesting a second carboxy-salicyl entity attached to the tricyclododecadiene core at C-20. Additional correlations between C-1 (δ 158.0) and H-7 (δ 5.19 and 5.40) and H-1′ (δ 5.09/5.07) and also between C-28 (δ 157.7) to H-22 (δ 5.16 and 5.38) and H-1″ (δ 5.07/5.09) were consistent with placement of the O-glucosides at C-1 and C-28.

### Miyabeacin B

For the initial isolation of Miyabeacin B, 2.5 mL of water:methanol (80:20) was added to Salix miyabeana stem tissue (200 mg). The suspension was agitated for 5 minutes at room temperature and then heated to 50° C. for 10 minutes using a water bath. The resultant solution was centrifuged at 13,000 rpm for 5 minutes. 800 μL of the supernatant, removed to a clean tube, was heated at 90° C. for 2 minutes. The solution was cooled (5° C.) for 30 minutes and centrifuged at 13,000 rpm for 5 minutes. The supernatant, containing the target compound was subjected to purification using reversed-phase HPLC. Injections of 100 μL each were made into an analytical HPLC using an Agilent 1100 HPLC system equipped with a quaternary pump, diode array detector, column oven and auto sampler. Peaks were separated using an Ascentis C18 column (5 um, 5×250 mm (Supelco, UK). The operating solvents were: Solvent A: H₂O with 0.1% formic acid, solvent B: acetonitrile with 0.1% formic acid. The operating gradient for peak isolation was from 5% B (0-10 min), 29% B (10-60 min) to 29% B (60-70 min) at a constant flow of 1 mL/min and using a total chromatographic run of 70 min. Peaks were identified and monitored using a wavelength of 254 nm and were collected manually into glass tubes. Miyabeacin B eluted at 52.11 min (FIG. 12). Equivalent fractions from multiple runs were combined and evaporated using a Speedvac concentrator (Genevac, Suffolk, UK) to yield 0.67 mg of purified Miyabeacin B. The structure of Miyabeacin B was determined by various forms of spectroscopy.

### Miyabeacin B Spectroscopic Analyses

High Resolution LC-MS: LC-MS was carried out in negative ion mode using a C18 column. Conditions of analysis are outlined in Table 3. FIG. 13 shows a total ion chromatogram of purified miyabeacin B which appeared as a single peak at 24.34 min. A high-resolution mass spectrum (FIG. 14) was collected in negative ion mode and showed an m/z ion at 843.23474 (C₄₀H₄₃O₂₀), corresponding to the [M-H]− of miyabeacin B (molecular formula C₄₀H₄₄O₂₀). A smaller ion at m/z 889.23895 (C₄₁H₄₅O₂₂) corresponded to the formate adduct.

NMR spectroscopy: 1H-NMR spectroscopy in aqueous methanol showed a total of 17 signals which related to 34 separate protons (Table 6).

TABLE 6
¹H-NMR assignments for miyabeacin B. Data collected at 600
MHz in D₂O:CD₃OD (4:1), referenced to d₄-TSP (0.01% w/v).

| Position | | |
|---|---|---|
| 1 | — | |
| 2 | 7.20 | (d, 8.2) |
| 3 | 7.43 | (ddd, 8.5, 7.5, 1.5) |
| 4 | 7.12 | (ddd, 7.5, 7.4, 0.9) |
| 5 | 7.35 | (dd, 7.5, 1.5) |
| 6 | — | |
| 7α | 5.46 | (d, 11.7) |
| 7β | 5.13 | (d, 11.7) |
| 8 | — | |
| 9 | — | |
| 10 | 2.76 | (dd, 4.4, 2.1) |
| 11 | 2.99 | (m) |
| 12 | 2.88 | (m) |
| 13 | 3.12 | (dd, 7.6, 4.0) |
| 14 | — | |
| 15 | 2.76 | (dd, 4.4, 2.1) |
| 16 | 2.99 | (m) |
| 17 | 2.88 | (m) |
| 18 | 3.12 | (dd, 7.6, 4.0) |
| 19 | — | |
| 20 | — | |
| 21 | — | |
| 22β | 5.46 | (d, 11.7) |
| 22α | 5.13 | (d, 11.7) |
| 23 | | |
| 24 | 7.35 | (dd, 7.5, 1.5) |
| 25 | 7.12 | (ddd, 7.5, 7.4, 0.9) |
| 26 | 7.43 | (ddd, 8.5, 7.5, 1.5) |
| 27 | 7.20 | (d, 8.2) |
| 28 | | |
| 1′ | 5.07 | (d, 7.8) |
| 2′ | 3.51 | (dd, 9.4, 7.8) |
| 3′ | 3.58 | (m) |
| 4′ | 3.45 | (t, 9.4) |
| 5′ | 3.58 | (m) |
| 6′β | 3.72 | (dd, 12.4, 6.0) |
| 6′α | 3.99 | (dd, 12.5, 2.2) |
| 1″ | 5.07 | (d, 7.8) |
| 2″ | 3.51 | (dd, 9.4, 7.8) |
| 3″ | 3.58 | (m) |
| 4″ | 3.45 | (t, 9.4) |
| 5″ | 3.58 | (m) |
| 6″β | 3.72 | (dd, 12.4, 6.0) |
| 6″α | 3.99 | (dd, 12.5, 2.2) |

The presence of signals relating to benzyl and glucosyl moieties compared well with those observed in the ¹H-NMR spectrum of miyabeacin. Absence of the four olefin signals (δ5.91 to 6.59) previously observed in miyabeacin was accompanied by a movement upfield of the four bridgehead protons (δ3.43-3.63) to give a set of four signals at 62.76, 2.88, 2.99 and 3.12 each integrating for 2 protons. The ¹H-NMR data suggested a further [2+2] intramolecular cyclization of the olefin units in miyabeacin to give a “caged” structure which we have named miyabeacin B. The cycloaddition of the double bonds in now confers a 2-fold axis of symmetry resulting in a significant simplification of the ¹H-NMR spectrum for miyabeacin B relative to that observed for miyabeacin. [¹H-¹H] correlation spectroscopy confirmed the linkages around the tricyclic core of the molecule. 13C data for miyabeacin B is given in Table 7.

TABLE 7
¹³C-NMR assignment for miyabeacin B. Data collected at 100.61
MHz in D₂O:CD₃OD (4:1), referenced to d₄-TSP (0.01% w/v).

| Position | |
|---|---|
| 1 | 158.1 |
| 2 | 117.9 |
| 3 | 133.9 |
| 4 | 125.9 |
| 5 | 134.3 |
| 6 | 126.4 |
| 7α | 67.1 |
| 7β | 67.1 |
| 8 | 174.0 |
| 9 | 80.6 |
| 10 | 43.1 |
| 11 | 41.0 |
| 12 | 37.7 |
| 13 | 48.1 |
| 14 | 210.6 |
| 15 | 43.1 |
| 16 | 41.0 |
| 17 | 37.7 |
| 18 | 48.1 |
| 19 | 210.6 |
| 20 | 80.6 |
| 21 | 174.0 |
| 22β | 67.1 |
| 22α | 67.1 |
| 23 | 126.4 |
| 24 | 134.3 |
| 25 | 125.9 |
| 26 | 133.9 |
| 27 | 117.9 |
| 28 | 158.1 |
| 1′ | 102.9 |
| 2′ | 76.0 |
| 3′ | 79.0 |
| 4′ | 72.8 |
| 5′ | 79.0 |
| 6′β | 63.9 |
| 6′α | 63.9 |
| 1″ | 102.9 |
| 2″ | 76.0 |
| 3″ | 79.0 |
| 4″ | 72.8 |
| 5″ | 79.0 |
| 6″β | 63.9 |
| 6″α | 63.9 |

### Miyabeanol

For the initial isolation of Miyabeanol, 2 mL of water:methanol (80:20) was added to Salix miyabeana leaf tissue (150 mg). The suspension was agitated for 5 minutes at room temperature and then heated to 50° C. for 10 minutes using a water bath. The resultant solution was centrifuged at 13,000 rpm for 5 minutes. 800 μL of the supernatant, removed to a clean tube, was heated at 90° C. for 2 minutes. The solution was cooled (5° C.) for 30 minutes and centrifuged at 13,000 rpm for 5 minutes. The supernatant, containing the target compound was subjected to purification using reversed-phase HPLC. 8 repeated injections of 100 μL each were made into an analytical HPLC using an Agilent 1100 HPLC system equipped with a quaternary pump, diode array detector, column oven and auto sampler. Peaks were separated using an Ascentis C18 column (Sum, 5×250 mm (Supelco, UK). The operating solvents were: Solvent A: H₂O with 0.1% formic acid, solvent B: acetonitrile with 0.1% formic acid. The operating gradient for peak isolation was from 5% B (0-10 min), 22% B (10-50 min) to 37% B (60-70 min) at a constant flow of 1 mL/min and using a total chromatographic run of 72 min. Peaks were identified and monitored using a wavelength of 254 nm and were collected manually into glass tubes. Miyabeanol eluted at 44.87 min (FIG. 1). Equivalent fractions from 8 runs were combined and evaporated using a Speedvac concentrator (Genevac, Suffolk, UK) to yield 1.05 mg of purified Miyabeanol.

### Miyabeanol Spectroscopic Analyses

High Resolution LC-MS: LC-MS was carried out in negative ion mode using a C18 column. Conditions of analysis are outlined in Table 3. FIG. 15 shows a total ion chromatogram of purified miyabeacin B which appeared as a single peak at 20.11 min. A high-resolution mass spectrum (FIG. 16) was collected in negative ion mode and showed an m/z ion at 531.15074(C₂₆H₂₇O₁₂), corresponding to the [M-H]− of miyabeanol (molecular formula C₂₆H₂₈O₁₂). Additional ions at 421.11421 (C₂₀H₂₁O₁₀) and 467.11943 (C₂₁H₂₃O₁₂) corresponded to the product of a reverse Diels Alder reaction (salicortenone) and its corresponding formate adduct. MSMS analysis of m/z 531.15074 (FIG. 17) gave rise to fragment ions at 245.04634 (C₁₃H₉O₅), 217.05150 (C₁₂H₉O₄) and 123.04579 (C₇H₇O₂).

NMR spectroscopy: The ¹H NMR spectrum of miyabeanol (FIG. 18 and Table 8) suggested an analogous structure to the cyclodimer miyabeacin, although certain regions of the spectrum, including those relating to the benzyl and glucosyl groups, were no longer duplicated suggesting that one of each of these units had been lost.

TABLE 8
¹H-NMR assignments for miyabeanol. Data collected at 600
MHz in D₂O:CD₃OD (4:1), referenced to d₄-TSP (0.01% w/v).

| Position | | |
|---|---|---|
| 1 | | |
| 2 | 7.19 | (d, 8.0) |
| 3 | 7.40 | (m) |
| 4 | 7.12 | (td, 7.5, 0.9) |
| 5 | 7.31 | (dd, 7.6, 1.5) |
| 6 | — | |
| 7α | 5.39 | (d, 11.9) |
| 7β | 5.18 | (d, 11.9) |
| 8 | — | |
| 9 | — | |
| 10 | 3.57-3.61 | (m) |
| 11 | 3.48-3.53 | (m) |
| 12 | 6.63 | (dd, 10.2, 4.1) |
| 13 | 6.02 | (dd, 10.1, 1.7) |
| 14 | — | |
| 15 | 3.28-3.33 | (m) |
| 16 | 6.27 | ( ddd, 7.9, 6.9, |
| 17 | 5.94 | (1H, ddd, 7.9, |
| 18 | 3.36 | (1H, ddd, 6.0, |
| 19 | — | |
| 20 | — | |
| 21 | — | |
| 22β | — | |
| 22α | | |
| 23 | | |
| 24 | | |
| 25 | | |
| 26 | | |
| 27 | | |
| 28 | | |
| 1′ | 5.06 | (d, 7.3) |
| 2′ | 3.49-3.59 | (m) |
| 3′ | 3.54-3.61 | (m) |
| 4′ | 3.45-3.53 | (m) |
| 5′ | 3.54-3.61 | (m) |
| 6′β | 3.76 | (dd, 12.5, 5.9) |
| 6′α | 3.92 | (dd, 12.4, 2.2) |

```
  1″
  2″
  3″
  4″
  5″
    6″β
    6″α
```

¹H signals at δ 6.63 and δ 6.02 corresponded to those observed in miyabeacin and related to the enone protons, H-12 and H-13. Signals corresponding to the isolated olefin protons at δ 6.27 and δ 5.94 were also present. This data and additional ¹H-¹H COSY correlations (FIG. 19) of these signals to 4 additional methine protons confirmed that the molecule retained the Diels-Alder “core”. 13C NMR (FIG. 20 and Table 9) showed 26 separate carbon signals including 2 ketone signals at δ 213.29 and δ 199.1.

TABLE 9
¹³C-NMR assignments for miyabeanol. Data collected at 100.61
MHz in D₂O:CD₃OD (4:1), referenced to d₄-TSP (0.01% w/v).

| | [7] | [7] |
|---|---|---|
| Position | (D2O:CD₃OD) | (D2O) |
| 1 | 158.0 | 157.8 |
| 2 | 117.7 | 117.3 |
| 3 | 133.7 | 133.2 |
| 4 | 125.7 | 125.3 |
| 5 | 133.7 | 133.2 |
| 6 | 126.6 | 126.6 |
| 7α | 67.2 | 67.0 |
| 7β | 67.2 | 67.0 |
| 8 | 173.7 | 173.6 |
| 9 | 82.5 | 82.4 |
| 10 | 40.6 | 40.4 |
| 11 | 43.9 | 43.7 |
| 12 | 152.8 | 152.7 |
| 13 | 130.8 | 130.6 |
| 14 | 199.0 | 199.1 |
| 15 | 45.7 | 45.5 |
| 16 | 136.0 | 135.6 |
| 17 | 132.2 | 132.2 |
| 18 | 54.6 | 54.5 |
| 19 | 213.3 | 213.4 |
| 20 | missing | 81.4 |
| 21 | — | — |
| 22β | — | — |
| 22α | — | — |
| 23 | — | — |
| 24 | — | — |
| 25 | — | — |
| 26 | — | — |
| 27 | — | — |
| 28 | — | — |
| 1′ | 103.0 | 102.7 |
| 2′ | 76.0 | 75.8 |
| 3′ | 79.1 | 78.7 |
| 4′ | 72.4 | 72.2 |
| 5′ | 78.8 | 78.5 |
| 6′β | 63.6 | 63.5 |
| 6′α | 63.6 | 63.5 |
| 1″ | — | — |
| 2″ | — | — |
| 3″ | — | — |
| 4″ | — | — |
| 5″ | — | — |
| 6″β | — | — |
| 6″α | — | — |

The position of side-chain loss and decarboxylation was confirmed via extensive analysis of COSY, HSQC and HMBC correlation spectroscopy. Key ¹H-¹³C correlations were identified between H-10 and C-8, H-10 to C-14 and H-13 to C-9. This allowed placement of the carboxy-salicylglycoside moiety at C-9. Correlations from H-15 and H-18 to the carbonyl at C-19 and from H-16, H-18 and H-10 to C-20 (δ81.4) were all present.

### Bioassay Data of Miyabeacin

The activity of miyabeacin was tested against a range of cancer cell lines including those in neuroblastoma and breast, oesophageal and ovarian cancers (Table 10).

TABLE 10
Bioactivity data of miyabeacin in six cancer cell lines

| | | Miyabeacin IC50 |
|---|---|---|
| cancer type | cell line | (μg/mL) |
| breast | BT-474 | 27.04 |
| oesophageal | COLO-680N | 5.08 |
| ovarian | COLO-704 | 20.18 |
| ovarian | EFO-21 | 12.69 |
| breast | MCF-7 | 2.19 |
| neuroblastoma | UKF-NB-3 | 7.12 |

The MYCN-amplified neuroblastoma cell line UKF-NB-3 was established from a stage 4 neuroblastoma patient (Kotchetkov et al., 2005). Also tested was the vincristine-resistant UKF-NB-3 sub-line UKF-NB-3^{r}VCR (Rothwell et al., 2010) (adapted to grow in the presence of vincristine 10 ng/mL). At a concentration of 20 μg/mL of miyabeacin, the cell viability, relative to non-treated cells, after 120 hours was 0% for UKF-NB-3 and 4.22±2.89% for the vincristine resistant UKF-NB-3^{r}VCR line. The oesophageal cancer cell line COLO-680N was obtained from ATCC (Manassas, Va., USA) and the ovarian cancer cell line COLO-704 from DSMZ (Braunschweig, Germany). All cell lines were propagated in Iscove's modified Dulbecco's medium (IMDM) supplemented with 10% FCS, 100 IU/ml penicillin and 100 mg/ml streptomycin at 37° C. Cells were routinely tested for mycoplasma contamination and authenticated by short tandem repeat profiling. Cell viability was determined by the 3-(4,5-dimethylthiazol-2-yl)-2,5-diphenyltetrazolium bromide (MTT) dye reduction assay after 120 h incubation as described previously (Michaelis et al., 2015). Briefly, 5000 cells (suspended in 100 μL IMDM supplemented with 10% FCS, 100 IU/ml penicillin and 100 mg/ml streptomycin) were incubated in 96-well plates at 37° C. and 5% CO₂ in the absence or presence of varying compound concentrations for 120 h. Then, 25 μL of MTT solution (2 μg/mL dissolved in PBS) were added for 4 h. This was followed by the addition of 100 Lp of 20% sodium dodecyl sulphate (50:50 purified water/DMF) solution adjusted to pH 4.7 for an additional 4 h in order to lyse cells and dissolve formazan precipitates. Plates were then read at 600 nm. The relative viability was determined as the relative reduction of the optical density relative to an untreated cell control (=100%). Replicated IC50 values for miyabeacin activity were determined on three selected lines (UKF-NB-3, COLO-680N and COLO-704) and ranged from 17.15 μM to 40.18 μM (Table 11).

TABLE 11
Replicated IC₅₀ determination in three cancer cell lines

| | | miyabeacin IC50 concentration | | | | | IC50 |
|---|---|---|---|---|---|---|---|
| | | (μg/mL) | | | | | (μM) |
| cancer type | cell line | expt 1 | expt 2 | expt 3 | mean | S.D. | mean |
| oesophageal | COLO- | 5.08 | 15.08 | 51.46 | 23.87 | 19.93 | 28.28 |
| cancer | 680N | | | | | | |
| ovarian cancer | COLO-704 | 20.18 | 32.38 | 50.79 | 34.45 | 12.58 | 40.18 |
| neuroblastoma | UKF-NB-3 | 7.12 | 20.97 | 15.33 | 14.47 | 5.69 | 17.15 |

Whilst the results are important for all of the cells lines shown above, of particular note is the activity against neuroblastoma cell lines. Overall survival rates are below 50% and it represents the most frequent extracranial solid childhood tumour. With resistance acquisition being a significant issue in neuroblastoma, new compounds effective against neuroblastoma are in great need.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

### REFERENCES

Kotchetkov R, Driever P H, Cinatl J, Michaelis M, Karaskova J, Blaheta R, Squire J A, Von Deimling A, Moog J, Cinatl J Jr. Increased malignant behavior in neuroblastoma cells with acquired multi-drug resistance does not depend on P-gp expression. Int J Oncol. 2005 October; 27(4):1029-37.
Michaelis M, Rothweiler F, Barth S, Cinatl J, van Rikxoort M, Loschmann N, Voges Y, Breitling R, von Deimling A, Rödel F, Weber K, Fehse B, Mack E, Stiewe T, Doerr H W, Speidel D, Cinatl J Jr. Adaptation of cancer cells from different entities to the MDM2 inhibitor nutlin-3 results in the emergence of p53-mutated multi-drug-resistant cancer cells. Cell Death Dis. 2011 Dec. 15; 2:e243.
Michaelis M, Agha B, Rothweiler F, Loschmann N, Voges Y, Mittelbronn M, Starzetz T, Harter P N, Abhari B A, Fulda S, Westermann F, Riecken K, Spek S, Langer K, Wiese M, Dirks W G, Zehner R, Cinatl J, Wass M N, Cinatl J Jr. Identification of flubendazole as potential anti-neuroblastoma compound in a large cell line screen. Sci Rep. 2015a Feb. 3; 5:8202.
Rothwell, P. M., et al. Long-term effect of aspirin on colorectal cancer incidence and mortality: 20-year follow-up of five randomised trials. Lancet 376:1741-50 (2010).

The content of all references cited herein are incorporated herein by reference in their entirety.

1. (canceled)

2. A compound of Formula I, or a derivative, homologue, stereoisomer, prodrug or pharmaceutical salt thereof,
R¹-L-R² Formula I
wherein L is a linking member and, R¹ and R² are each independently selected from Formula III, 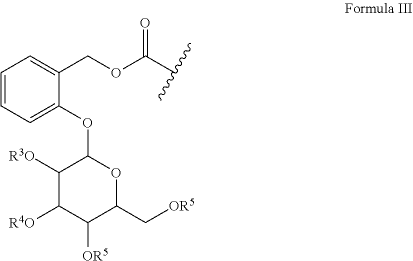
wherein R³, R⁴, R⁵ and R⁶ are each independently selected from (i) H, (ii) acetyl, (iii) benzoyl, (iv) ortho- or para-coumaroyl, (v) cinnamoyl,
wherein L is selected from
(a) Formula IIA:— 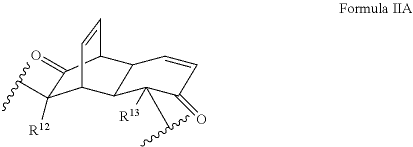
wherein R¹² and R¹³ are each independently selected from H and OH, or
(b) Formula IIB:— 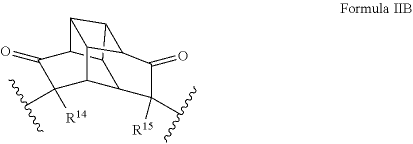
wherein R¹⁴ and R¹⁵ are each independently selected from H and OH.

3. The compound according to claim 2, wherein
(a) R¹ is Formula IIIA:— 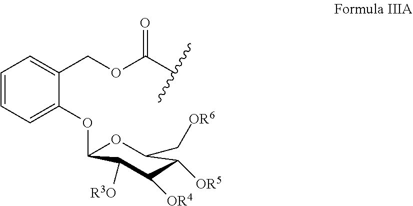
wherein R³, R⁴, R⁵ and R⁶ are each independently selected from (i) H, (ii) acetyl, (iii) benzoyl, (iv) ortho- or para-coumaroyl, (v) cinnamoyl, or
(b) R¹ is Formula IIIB:— 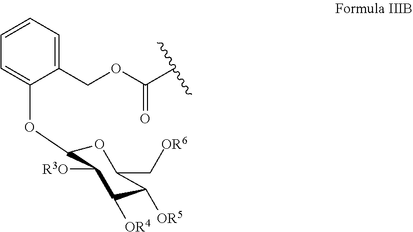
wherein R³, R⁴, R⁵ and R⁶ are each independently selected from (i) H, (ii) acetyl, (iii) benzoyl, (iv) ortho- or para-coumaroyl, (v) cinnamoyl.

4. The compound according to claim 2, wherein
(a) R² is Formula IIIC:— 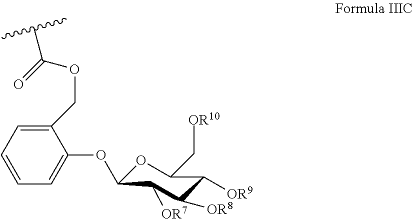
wherein R⁷, R⁸, R⁹ and R¹⁰ are each independently selected from (i) H, (ii) acetyl, (iii) benzoyl, (iv) ortho- or para-coumaroyl, (v) cinnamoyl, or
(b) R² is Formula IIID:— 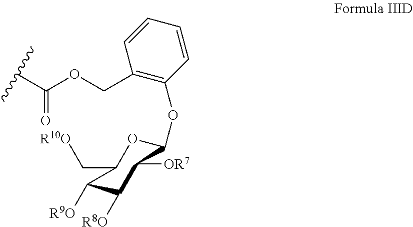
wherein R⁷, R⁸, R⁹ and R¹⁰ are each independently selected from (i) H, (ii) acetyl, (iii) benzoyl, (iv) ortho- or para-coumaroyl, (v) cinnamoyl.

5. (canceled)

6. (canceled)

7. (canceled)

8. (canceled)

9. The compound according to claim 1 selected from
(a) Formula VII or a derivative, homologue, stereoisomer, prodrug or pharmaceutical salt thereof, 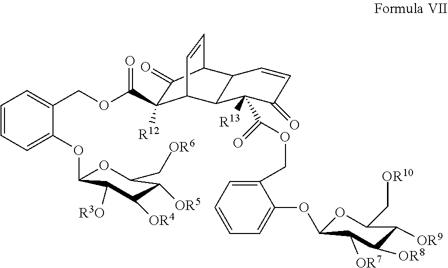
wherein R³, R⁴, R⁵, R⁶, R⁷, R⁸, R⁹ and R¹⁰ are each independently selected from (i) H, (ii) acetyl, (iii) benzoyl, (iv) ortho or para-coumaroyl, (v) cinnamoyl, and
wherein R¹² and R¹³ are each independently selected from H and OH, or
(b) Formula VIII or a derivative, homologue, stereoisomer, prodrug or pharmaceutical salt thereof, 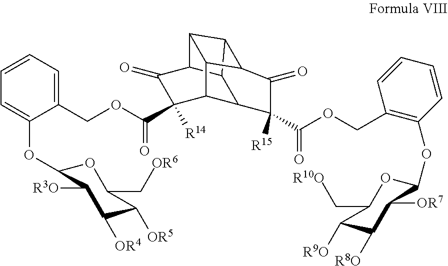
wherein R³, R⁴, R⁵, R⁶, R⁷, R⁸, R⁹ and R¹⁰ are each independently selected from (i) H, (ii) acetyl, (iii) benzoyl, (iv) ortho- or para-coumaroyl, (v) cinnamoyl, and
wherein R¹⁴ and R¹⁵ are each independently selected from H and OH.

10. The compound according to claim 9, wherein R³, R⁴, R⁵, R⁶, R⁷, R⁸, R⁹ and R¹⁰ are each independently selected from (i) H, and (ii) acetyl.

11. (canceled)

12. (canceled)

13. The compound according to claim 1 of Formula X or XI, or a derivative, homologue, stereoisomer, prodrug or pharmaceutical salt thereof.

14. A composition comprising a compound according to claim 1.

15. The composition according to claim 14, wherein the composition comprises a compound of Formula VII or VIII, or a derivative, homologue, stereoisomer, prodrug or pharmaceutical salt thereof.

16. The composition according to claim 14, wherein the composition comprises a compound of Formula X or XI.

17. The composition according to claim 14, wherein the composition comprises a compound of Formula X, or a derivative, homologue, stereoisomer, prodrug or pharmaceutical salt thereof,

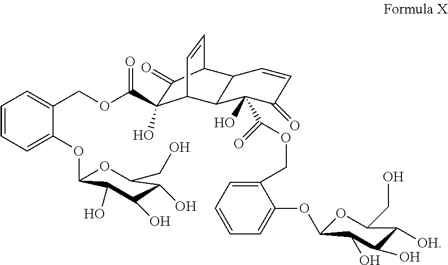

18. (canceled)

19. (canceled)

20. (canceled)

21. A method of treating a disease, wherein the method comprises administering to a patient suffering from a disease a therapeutically effective amount of a composition according to claim 14.

22. The method of claim 21, wherein the disease is cancer.

23. The method according to claim 22, wherein the cancer is selected from neuroblastoma, breast cancer, oesophageal cancer, or ovarian cancer.

24. (canceled)

25. (canceled)

26. A method of claim 22, wherein the cancer is primary or secondary (metastatic) cancer.

27. A method of claim 22, wherein the cancer is a drug-resistant cancer.
